(19) United States
(12) Patent Application Publication
(10) Pub. No.: US 2004/0242854 A1
(43) Pub. Date: Dec. 2, 2004

(54) Chemical capture reagent

(76) Inventors: Nigel John Osborn, Buckinghamshire (GB); Karen Williams, Buckinghamshire (GB); Alison Claire Sweet, Buckinghamshire (GB)

Correspondence Address: AMERSHAM BIOSCIENCES, PATENT DEPARTMENT, 800 CENTENNIAL AVENUE, PISCATAWAY, NJ, 08855, US

(21) Appl. No.: 10/482,715

(22) PCT Filed: Jul. 1, 2002
(86) PCT No.: PCT/GB02/03035

(30) Foreign Application Priority Data
Jul. 2, 2001 (GB) 0116143.9

(51) Int. Cl. C07K 14/00

(52) U.S. Cl. 530/409; 530/408

(57) ABSTRACT

Disclosed is a capture reagent having Formula I:

PRG-L-X-carrier (I)

wherein:

PRG is a peptide or protein reactive group;

L is a linker group comprising a stable isotopic detectable label moiety; and

X is a cleavable group.

Also disclosed is a method for selective labelling and purification of proteins and peptides comprising using a capture reagent of Formula I.

### TECHNICAL FIELD

The present invention relates to a chemical capture reagent for proteins and peptides. In particular, the invention relates to using a chemical capture reagent in methods of selective labelling and capture of peptides or proteins from samples prior to comparative quantification and analysis.

### BACKGROUND TO THE INVENTION

It is increasingly being recognised that interpretation of the wealth of new data arising from genomics requires a parallel understanding of the dynamics and differential expression of proteins and peptides themselves. Recent advances in proteomics have focused on developing high throughput methods for rapidly separating and analysing complex protein samples. The most common implementation of proteome analysis is based on the separation of complex protein samples by two-dimensional gel electrophoresis (2DE) and the subsequent sequential identification of peptides, after proteolysis of the separated protein species, by mass spectrometric (MS) techniques.

Although 2DE has considerable resolving power, it has not so far been possible to use this technology to display an entire cell or tissue proteome in a single experiment. Several classes of proteins have proven especially resistant to analysis by 2DE, including low and high molecular mass proteins, membrane proteins, proteins with extreme isoelectric points (pIs) and low abundance, e.g. regulatory, proteins (Corthals et al. (2000) Electrophoresis, 21:1104-1115). Some of these proteins escape detection unless prior enrichment steps are taken and this limits the dynamic range of the technique.

In addition, the sequential manner in which samples are processed in 2DE-based techniques limits sample throughput and makes automation difficult. Furthermore, the ability to compare differential protein expression by comparative quantitation of proteomes from different samples remains a major challenge in proteomics research.

Accordingly, there is a need for improved techniques which allow the selective labeling and purification of proteins and peptides to facilitate comparative analysis and quantitation.

WO 00/11208 describes a method for differential analysis which involves labeling the protein or peptide samples in solution with an isotopically-coded affinity tag (ICAT™). To compare protein abundance between two proteomes quantitatively, one proteome is treated with a ‘light’ (non-deuterated) version of the affinity reagent and a second proteome is treated with a ‘heavy’ (deuterated) version of this probe. Following trypsin digestion, the affinity reagent-labelled peptides are isolated by (strept)avidin chromatography and analysed by liquid chromatography MS. The ICAT™-affinity reagent (˜500 Da) is a relatively large modification that remains on each peptide throughout the MS analysis.

However, in order to analyse the isolated proteins or peptides, the strong affinity bond between the ICAT™ reagent and solid phase must be disrupted. Disrupting such affinity interactions often requires forcing conditions, which may cause damage to the protein or peptide sample prior to analysis or may result in low recovery yields of the sample. Moreover, the affinity reagent must be added in excess to ensure that all the protein is labelled and excess affinity support must be present to ensure that all labelled protein then binds. This method therefore requires a high level of sample manipulation with two separate steps to label and capture the desired proteins/peptides. Furthermore, where the affinity approach is used, non-specific binding between proteins/peptides in the sample and the affinity reagent on the solid surface may occur. In addition, endogenous biotin binding proteins in the sample may bind directly to a streptavidin-coated solid surface. This may give false results in an analysis and complicate the binding reaction.

WO 01/86306 describes a method for differential analysis of proteins. The method involves fragmenting differentially expressed protein samples, either enzymatically or chemically, to produce a peptide pool. A proportion of this peptide pool is then isotopically labelled, for instance on cysteine using a thiol-reactive reagent coupled to an affinity reagent such as biotin. The resultant peptides are then captured using a capture moiety and subsequently analysed by mass spectrometry. Mention is also made of the possibility of selectively labelling the N—, C— or both termini.

Zhou et al. (Nature Biotechnology (2002) 19, 512-515) describes methodology for differential analysis of a protein reactive group coupled to a linker itself bound to aminopropyl glass beads via a photochemically cleavable group. The paper describes only the use of cleavage via photochemical means.

Munchbach et al. (Analytical Chemistry (2000), 72, 4047-4057) describes an alternative strategy for differential analysis of proteins. Blocking of the lysine residues is achieved using succinic anhydride, followed by proteolysis, to liberate peptides whose N-termini can be subsequently labelled with either H4 or D4 1-(nicotinoyloxy)succinimide and the two samples then combined. The presence of the positive charge on labelled proteins under acidic conditions aids subsequent analysis by mass spectrometry.

U.S. Pat. No. 4,331,590 describes a binding assay for determination of the concentration of a ligand in a liquid medium based on a conjugate of the form glycone-dye indicator-ligand. Subsequent determination of the label is achieved after enzymatic cleavage of the glycosidic linkage between the glycone and the dye moiety resulting in an increase in the fluorescence. Separation of bound from non-bound components is achievable using a variety of different methods such as a solid-phase bound antibody as well as the use of immune complex precipitating agents and adsorbents. The separation is by affinity means rather than covalent.

An alternative strategy was devised by Smith (Analytical Chemistry (2001) 73, 2132-2139) where ¹⁵N-metabolic labelling was used to introduce labels into proteins. Iodoacetyl-poly ethylene oxide-biotin was then used to label the cysteine-containing proteins, the sample then being hydrolysed by trypsin. Cysteine-containing peptides were then isolated using immobilised avidin.

WO 02/42427 describes the use of protein mass tag reagents (PMT) that allow for selective isolation of either peptides or protein fragments from complex mixtures either with or without the digestion of the proteins. The PMT consists of at least an amino acid reactive moiety plus possibly one or more accessory moieties and/or one or more recognition moieties, the mass difference part of the compound being present at the amino acid reactive, accessory or recognition moiety. The accessory moiety may be a fluorescent chemical functionality or an entity that enhances separation of the proteins or peptides by the PMT reagents. In addition the labelling of proteins after proteolysis with a biotin-moiety containing an isotopic label is discussed.

Many of the methods in the art depend upon the use of affinity reagents (e.g. WO 00/11208, WO 01/86306, U.S. Pat. No. 4,331,590, WO 02/42427) for the capture and/or purification of peptides or proteins. The use of such affinity reagents necessitates additional handling/purification steps and may lead to binding of endogenous biotin containing peptides (e.g. acetyl CoA carboxylase) and non-specific adsorption. Indeed, quantitative cleavage of biotin-labelled peptides from affinity columns can prove difficult. The disruption of affinity bonds in such methods, and the use of photochemical energy in the Zhou method described above, may also cause protein/peptide damage. Furthermore, few of these methods are amenable to the analysis of hydrophobic proteins

There is therefore a need for an improved method.

The present invention involves the capture of proteins and/or peptides onto a carrier, such as a solid support, using a reagent which is covalently bonded to immobilise it onto the carrier and has a protein reactive group itself covalently connected to a linker containing a label, and a cleavable group. This allows the labelling and capture step to be combined in a single step by incubating the protein or peptide with the single reagent and provides protein or peptide attachment to a solid phase by covalent, chemical means (so-called “chemical capture”) rather than through an affinity interaction.

For the analysis of multiple protein samples in a differential approach, it is desirable to minimise the manipulations of each sample to maintain the original protein representation. Using the chemical capture approach of the present invention, the individual sample manipulation is reduced, requiring only a single step to label and capture the desired proteins/peptides by mixing each sample with the relevant solid support-bound reagent. The captured samples can then be combined, enabling all subsequent steps to be carried out on the mixed solid-support samples. This ensures that uniform treatment is applied to the two (or more) samples such that any differences observed are as a result of different protein expression levels and turnover rates in the samples rather than as a result of variations in the processing of the individual samples.

For MS analysis, as the chemical capture reagent relies on a covalent linkage with the solid phase and a cleavable linker rather than an affinity interaction, the molecular weight of the peptide and label together can be reduced because the sample is cleaved from the solid support leaving only the label attached to the protein/peptide, compared to an affinity approach where the labelled sample also includes the mass of the affinity group itself. This is advantageous as the resolution of MALDI and electrospray mass spectrometers is generally higher at lower molecular weight, thus a smaller label modification is preferable.

### SUMMARY OF INVENTION

The present invention provides a capture reagent having Formula (I):

PRG-L-X-carrier (I)

wherein:

PRG is a peptide or protein reactive group

L is a linker group

X is a cleavable group

Suitably, PRG may be any functional group that reacts selectively with any functional groups present in specific peptides or proteins. In a particularly preferred embodiment, the functional group targeted is cysteine residues and the PRG is selected from the group consisting of maleimide, pyridyldisulfide, vinylsulfone, iodoacetamide, epoxide, nitrile, aryl thiol, or sulfonated alkyl, in order to capture thiol containing proteins or peptides. In a particularly preferred embodiment the PRG is maleimide.

Several other functionalities exist on a protein or peptide that could also be specifically targeted e.g. ε-amino groups on lysine residues or the N-terminus of a peptide or protein could be targeted by active esters, such as N-hydroxysuccinimide ester, by isothiocyanates, isocyanates, imidoesters or sulphonyl halides as the PRG. Amines may also be specifically targeted with aldehydes and ketones in the presence of a reducing agent such as sodium borohydride or sodium cyanoborohydride.

Carboxylic acid residues on amino acids, such as glutamic acid or aspartic acid, or the C-terminus of an amino acid may be coupled to either amines or alcohols, for example using water soluble carbodiimides, such as the carbodiimide 1-ethyl-3-(3-dimethylaminopropyl)carbodiimide hydrochloride (EDAC) with or without the presence of catalysts such as 4-dimethylaminopyridine.

Phosphate-containing peptides (e.g. phosphoserine or phosphothreonine-containing peptides) can be modified by first treating them with barium or sodium hydroxide (see, for example, Oda et al (2001) Nature Biotech, 19, 379-382 and Byford MF, Biochem J, (1991) 280, 261-265) to effect elimination of the phosphate groups to leave an alkene group. The resultant alkene can be reacted directly with a carrier thiol functionality as the PRG or further treated with an excess of ethane dithiol to form a thiol group for binding to a maleimide or other thiol-reactive PRG.

For carbohydrate-containing proteins or peptides, suitable PRGs include amines or hydrazines which may react with aldehydes or ketones present on the carbohydrate with or without the presence of a dehydrating agent such as 2,2-dimethoxypropane followed by reduction of the resultant Schiff base with NaBH₄, or NaCNBH₃. Alternatively, oxidation of vicinal diols on the carbohydrate can be achieved, for example, with periodic acid and the resultant aldehyde coupled to an amine- or hydrazine-based linker.

The PRG may be chosen so as to covalently target a reaction with specific protein functions such as enzyme classes. For example, PRG may be based on inhibitors of specific enzyme classes such as kinases, phosphatases or proteases. Thus, in one embodiment, PRG may be based on a mechanism-based or suicide inhibitor. Such inhibitors (reviewed for example in Sandler et al. “Design of enzyme inhibitors”, Oxford Science Publications 1989 (ISBN 0-19-261537-8) and Walsh (1984) Ann. Rev. Biochem 53, 493-535) can display remarkable specificity for a particular enzyme or class of enzyme. For example, serine hydrolases are potently inhibited as a class of enzymes by fluorophosphonates as well as groups bearing α-halo or (acyloxy)methyl ketone substituents (Cravatt & Sorensen (2000), Curr. Opin Chem Biol., 4:663). Tyrosine kinase can be inhibited with 4-(phenylamino)quinazoline- and 4-(phenylamino)pyrido (Smaill et al., (2000) J Med Chem. 43(7), 1380-1397). Binding interactions between a PRG and different classes of enzymes are described, for example, by Cravatt and Sorensen. In another embodiment, PRG may be an enzyme substrate that is selectively cleaved or modified by the enzyme of interest rendering the enzyme bound to the capture reagent.

L is a linker group which is, preferably, substantially unreactive to peptides and proteins and to cleavage conditions. Suitable linker groups include: ethers, polyethers, amides, polyamides, polythioethers, disulfides, silyl ethers, methyl, alkyl or alkenyl chains (straight chain or branched and portions of which may be cyclic) aryl, diaryl or alkyl-aryl groups. In the context of the present invention, the term alkyl is to be interpreted as comprising 2 to 20 carbon atoms, preferably 2 to 10 carbon atoms. More preferably, the linker group L is a C₄ or a C₆ alkyl group. Aryl groups in linkers can contain one or more heteroatoms (e.g. N, O or S atoms).

Suitably cleavage of the cleavable group results in the detectable label moiety remaining bound to the PRG and the captured protein or peptide.

In one embodiment, at least some of the atoms in the linker or the cleavable group may be readily replaced with stable heavy isotopes, for example, hydrogen may be replaced by deuterium. Different combinations of hydrogens and deuteriums may be used to enable multiple samples to be differentially labelled and detected by mass spectrometry. Suitably, at least 2 deuteriums but preferably 6 to 8 deuteriums are included where mass spectrometric detection is used.

Particularly preferred linkers using stable heavy atoms include those given by the following Formulae V-IX in which H/D indicates where the Hydrogen atoms (H) may be readily replaced with Deuterium (D).

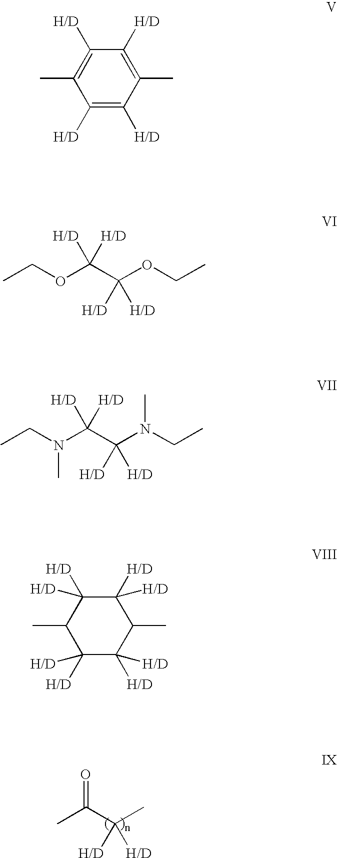

Suitable label moieties include stable isotopic labels such as ²H, ¹³C, ¹⁵N, ¹⁷O, ¹⁸O, ³⁴S, ³³S, ³⁶S.

Suitably, the detectable label exists in at least two distinguishable forms enabling quantitative differential analysis between two or more samples to be performed. In a particularly preferred embodiment, the distinguishable forms have substantially the same chemical structure such that two samples labelled with two distinguishably labelled capture reagents have substantially indistinguishable mobility when analysed by chromatography or other separation methods such as 2DE, capillary electrophoresis (CE).

In a particularly preferred embodiment for mass spectrometric detection, distinguishable labels would comprise differentially isotopically labelled linkers that give a mass difference that can be resolved in the mass spectrometer.

The linker may also comprise additional modifications to enhance ionisation in the mass spectrometer. Depending on the nature of the PRG, the modification may include the addition of an acidic or basic group; e.g., COOH, SO₃H, primary, secondary or tertiary amino groups, ethers, nitrogen-heterocycle or specific combinations of these groups can be employed in the linker to promote ionisation. Alternatively, the linker may also contain groups having a permanent charge, e.g., phosphonium groups, quaternary ammonium groups, tetralkyl or tetraryl borate, trityl, sulfonium groups, chelated-metal ions, or stable carbanions to enhance ionisation of the labelled species. Other suitable linker groups include trityl (triphenylmethyl) groups described, for example, in WO99/6007.

X is a chemical group that may be cleaved specifically to release the protein/peptide sample from the carrier molecule whilst leaving the label attached to the sample.

Cleavage may be achieved by a number of different strategies, notably by acidic or basic treatment, by photochemical or thermal means, by enzymatic cleavage, by electrochemical, reductive, nucleophilic or electrophilic cleavage. Suitably, the cleavable group, X, is positioned such that, following a cleavage reaction, the label in the linker group, L, remains bound to the captured protein or peptide. It will be understood that following protein or peptide capture, cleavage may result in X remaining attached either to the carrier (i.e. the L-X bond being cleaved) or to the linker group L (i.e. the X-carrier bond being cleaved).

A number of different cleavable functionalities could be envisaged. For example, acid cleavable groups have been described by Floersheimer (Peptides, p131-132, 1991) and Mergler (Tetrahedron Letts. (1998) 29, 4005-4012). These are cleavable using 1% trifluoroacetic acid in a suitable solvent. Other acid labile groups have been described by Albericio (Tetrahedron Letts. (1991) 32, 1015-1018) and include groups which are cleavable with 0.1% trifluoroacetic acid. A similar approach to linker cleavage was employed by Rink (Tetrahedron Letts. (1987) 28, 3787-3790) where the labile group is cleavable in 10% acetic acid.

Base labile linkage groups have been described (Liu, Int J Pept Protein Res. (1990) 35, 95-98) together with the cleavable group described by Albericio (Tetrahedron Letts. (1991) 32, 1515-1518) which cleaves through a β-elimination process using piperidine or diazabicyclo-[5.4.0]undec-5-ene (DBU).

Groups cleavable with fluoride ions have also been developed and are described, for example by Ramage (Tetrahedron (1992) 48, 499-514) and Mullen (Tetrahedron (1987) 28; 491-494). Reductive cleavage of the group is possible with ammonium formate/palladium catalysed hydrogenolysis (see, for example, Anwer (1981) Tetrahedron Letts., 22, 4369-4372) whereas reductive cleavage of a 2-azidomethyl-4-hydroxy-6,N-dimethylbenzamide moiety requires triphenylphosphine (Robinson (1993) Tetrahedron 49, 2873-2884).

A nitrobenzophenone based cleavable group such as that described by Findeis (J Org Chem. (1989) 54, 3478-3482) and Kaiser (Science (1989) 243, 187-191) can be cleaved nucleophilically using amines, hydrazine and carboxylic acids.

In another embodiment, for mass spectrometric detection the cleavable group itself may be differentially labelled using heavy atoms, such as deuterium, or stable isotopes such that cleavage results in the label remaining bound to the captured protein or peptide.

Suitably the cleavable group, X, is covalently attached to the carrier which comprises, or may be functionalised to comprise, a reactive group. For example, X may comprise an activated carboxylic acid (such as NHS ester) while the carrier may comprise an amine (or vice versa). Other suitable pairs which can form covalent bonds in this way include epoxide—nucleophile (e.g. amine, thiol or alcohol) to form an ether linkage, maleimide—thiol and boronic acid—1,2-diol (e.g. 1, 2—dihydroxyethane and other dihydroxyalkanes). In a particularly preferred embodiment, the cleavable group, X, is attached to the carrier via 1-carboxymethyl-3-formyl indole.

It is preferred that the components of the compound of Formula I are selected to enable peptide or protein capture to occur such that peptide/protein coupling to the PRG can occur under conditions where the cleavable moiety X is stable. Cleavage at X can occur under conditions that leave L-PRG-peptide/protein intact. Suitably, where a hydrogen/deuterium label is present, the reaction conditions must be such that there is no detectable proton exchange reaction between exchangeable protons in the linker group and the solvent during any of the sample manipulations.

In one embodiment, the carrier may be a solid support. Suitably the solid support is particulate i.e. of a nature suitable to give a high surface area for protein/peptide capture.

The solid support could be a number of different types, with varying functionalities. For example Sepharose (e.g. Sepharose 6B) based solid supports containing carboxy, amine, thiol, epoxy, cyanogen bromide, hydrazide and N-hydroxysuccinimide esters as the coupling functionality (e.g. NHS-activated Sepharose, CNBr-activated Sepharose (Amersham Biosciences). Toyopearl epoxy resin (TosoHaas) or Controlled Pore Glass (CPG) with aminopropyl, aminoaryl, N-hydroxysuccinimide ester, glyceryl, hydrazide, alkylamine, thiopropyl functionality present may also be employed. The pore size of the solid support could be used to discriminate between various sizes of protein. Silica-based solid supports e.g. CombiZorb S (Agilent Technologies) are functionailised with either a triamine or monoamine which could be used as a handle for further derivatisation.

Suitably, the density of protein reactive groups on the solid phase is such that an excess of solid phase-bound PRGs may be used to ensure that the reaction kinetics are in favour of bound peptide, with any excess PRG being quenched once the reaction has gone to completion. The loading of the solid support with the protein reactive groups is dependent on the starting density of functional group on the solid support used to prepare the chemical capture reagent. The density or substitution level of a functional group on the solid support is generally quoted in μmoles per gram of dry solid support. Substitution levels for functional groups vary widely from around 800 μmol/g for Toyopearl epoxy resin to 19 μmol/g for epoxy functionalised Sepharose 6B. However, where the PRG on the solid support is thiol reactive, targeting proteolytic digests; then only a small proportion of the peptides would be expected to bind given that less than 5% of amino acids are cysteine. Accordingly, in one preferred embodiment, the density of the functional groups on the carrier is in the range of 1 to 100 μmol/g.

In addition magnetic beads, latex, agarose or any biopolymer could also be envisaged as potential supports for the chemistry. Chemisorption can also be applied to the immobilisation of biopolymers on a surface (Bioconjugation, 1^{st} Edition, Dent and Aslam; 531-553). A large number of different functionalities, listed in this publication, are available commercially.

In another embodiment, the carrier may be a water soluble carrier or macromolecule which is also, preferably, chemically stable. Suitable soluble macromolecules include polyethylene glycol (PEG), such as PEG-based solid supports containing N-hydroxysuccinimide ester, aldehydes, maleimides, and MPEG-BTC (Harris, Poly(Ethylene Glycol) Chemistry: Biotechnical and Biomedical Applications p1-14 Plenum press). Other suitable soluble macromolecules include copolymers of HPMA (N-(2-hydroxypropyl) methyacrylamide) and acrylamide-based molecules with PEG linkers. Still further water soluble macromolecules include Dextran T-40 (Amersham Biosciences), Ficoll PM70 (Amersham Biosciences), Polyethylene glycol (Fluka), Polypropylene glycol (Fluka), Poly-L-lysine hydrobromide (Fluka), Polyvinyl alcohol (Fluka), Chitosan (Aldrich), Polyethylenimine (Aldrich), Polyallylamine (Aldrich), Poly(dimethylamine-co-epichlorohydrin) (Aldrich), Starburst PAMAM dendrimers (Aldrich) and DAB-Am polypropylenimine dendrimers (Aldrich). In a preferred embodiment, the water soluble linker comprises Ficoll PM70.

The advantage of using such a soluble macromolecule is that it is more compatible with chromatographic separations for washing to remove uncaptured peptides and elution after cleavage.

In a particularly preferred embodiment, there is provided a capture reagent of Formula II:

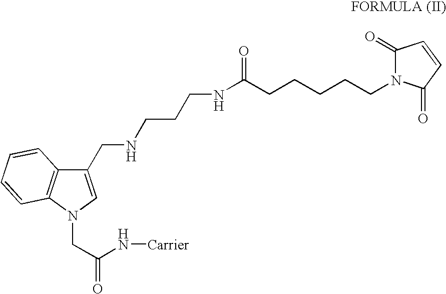

In another preferred embodiment, there is provided a capture reagent of Formula III:

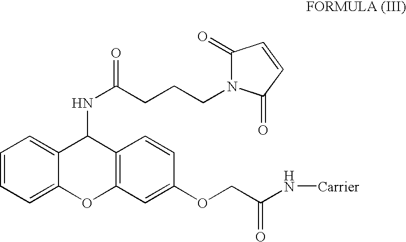

In a further preferred embodiment, there is provided a capture reagent of Formula IV:

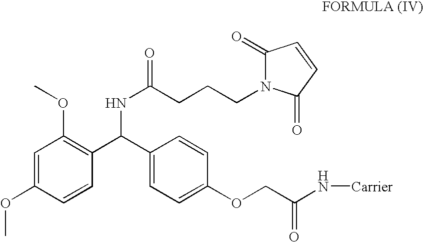

In a second aspect of the invention, a method for making a capture reagent in accordance with the first aspect is provided. The method of generating a capture reagent PRG-L-X-carrier can be achieved in several ways. Suitably, said method may comprise first synthesising a compound having the formula PRG-L-X prior to coupling it directly with a functionality on the carrier. Alternatively, the method may comprise attachment of the cleavable group, X, onto the carrier followed by sequential addition of the groups L-PRG or L, or PRG. It is important to cap excess amino functionalities on the carrier molecule prior to the removal of the protecting group.

In a third aspect of the invention there is provided a method for selective labelling and purification of proteins and peptides comprising the steps of:

a) providing a capture reagent in accordance with the first aspect of the invention to a sample containing proteins or peptides under conditions to promote attachment of the proteins or peptides to the PRG;

b) releasing captured components from the carrier; and

c) detecting and identifying the released components.

In a fourth aspect of the invention, there is provided a method for selective labelling and purification of proteins and peptides in one or more samples containing mixtures of proteins or peptides comprising the steps of:

a) providing a capture reagent in accordance with the first aspect of the invention to one or more samples containing proteins or peptides under conditions to promote attachment of the proteins or peptides to the PRG, wherein the detectable label of the capture reagent provided to one sample is distinguishable from that provided to another;

b) releasing captured components from the carrier; and

c) detecting and identifying the released components.

Optionally, the method further-comprises a pre-treatment step wherein functional groups on the protein or peptide are modified to render them reactive with the protein reactive group PRG prior to conducting step a) above. In one embodiment, the modification comprises treatment of carboxylic acid groups on the peptide or protein with a water soluble carbodiimide, such as the carbodiimide 1-ethyl-3-(3-dimethylaminopropyl) carbodiimide hydrochloride (EDAC) with or without the presence of catalysts such as 4-dimethylaminopyridine.

In another embodiment, the modification comprises elimination of phosphate groups on the peptide or protein to leave a reactive alkene group which, optionally, may be converted to a thiol group. The alkene group can be reacted directly with a thiol functionality as the PRG; if converted to a thiol group, this can in turn be bound to maleimide or other thiol-reactive PRG.

In the case of carbohydrate-containing proteins or peptides, the modification may comprise oxidation of vicinal diol groups or dehydration and reduction of aldehyde or ketone groups. These groups will react with amine or hydrazine functionalities of the PRG.

Suitably, the samples may be derived from multiple different protein samples, for example, different cell types, cells subjected to different treatments or healthy and diseased cells, in order to make a comparison of the proteome in the two or more samples.

Accordingly, in one embodiment of the fourth aspect the method further comprises the step:

d) measuring the relative abundances of the released components derived from each sample.

Suitably the method allows determination of differences in protein content of the samples such as relative expression levels of proteins.

Preferably, in either the third or fourth aspects of the invention, the proteins or peptide components in the samples are pre-treated, for example enzymatically (e.g. subjected to proteolytic degradation with trypsin) or chemically processed, before or after their capture. The samples may also-be fractionated prior to capture. In one embodiment, protein samples may be reduced or subjected to proteolysis prior to capture. In another embodiment, peptide components may be treated so as to ensure oxidation of N-terminal serine or threonine functionalities. In this embodiment, proteolytic degradation of protein samples is followed by, for example, periodate treatment. Oxidised samples can then be specifically coupled to a carrier with, for example, an aminooxy protein reactive group (Gaertner (1996) Bioconjugate Chemistry 7, 38-44).

In a particularly preferred embodiment captured components derived from different samples may be combined prior to releasing all captured components.

In another embodiment, proteins bound to the capture reagent are treated by enzymatic or chemical reaction and unbound peptides removed by washing. Preferably, washing to remove unreacted or non-specifically bound proteins or peptides can take place before or after mixing of the two samples.

The mode for releasing captured components from the carrier depends on the nature of the cleavable group, X. Preferably cleavage of the interaction is by chemical, pH, enzymatic or photochemical means.

In one embodiment of either the third or fourth aspect of the invention, an additional separation step can be performed before the detection step c). Suitably, the separation step may be by high performance liquid chromatography, e.g. on a C18 reverse phase column, ion exchange chromatography, gel filtration or hydrophobic interaction chromatography, or by electrophoretic methods e.g. capillary electrophoresis, 1D or 2D gel electrophoresis. The separation step may also possibly be a multidimensional or parallel purification.

Where the capture reagent comprises a fluorescent label moiety, the proteins labelled with fluors preferably would be separated by 2D or 1D electrophoresis followed by excision and digestion of proteins of interest, or by capillary electrophoresis.

It will be recognised that the method of detection in step c) depends on the nature of the label moiety which is included in the capture reagent. Preferably, the method of detection and identification of the released components in step c) is a mass spectrometric method such as MALDI or electrospray mass spectrometry. In another embodiment, the method of detection may be a fluorescent detection technique such as use of a CCD camera or fluorescent scanner or fluorescent lifetime.

Methods and instrument control protocols for the analysis of peptides by mass spectrometry are well-known in the art and described, for example, in Ducret et al. (Prot. Sci. (1998) 7, 706-719); Figeys & Aebersold (Electrophoresis (1998), 19, 885-892); Figeys et al. (Electrophoresis (1998), 19, 1811-1818); and Haynes et al. (Electrophoresis (1998) 19, 939-945).

Both the quantity and sequence identity of the proteins from which the tagged proteins originated can be determined by automated multistage MS. This is achieved by the operation of the mass spectrometer in a dual mode in which it alternates in successive scans between measuring the relative quantities of peptides eluting from the capillary column and recognising the sequence information of selected peptides. Peptides are quantified by measuring in the MS mode the relative signal intensities for pairs of peptide ions of identical sequence that are tagged with the isotopically light or heavy forms of the reagent, respectively, and which therefore differ in mass by the mass differential encoded within the tagged reagent. Peptide sequence information is automatically generated by selecting peptide ions of a particular mass to charge ratio for collision induced dissociation in the mass spectrometer. The resulting spectra are then automatically correlated with sequence databases to identify the protein from which the sequenced peptide originates. Combination of the results from MS analyses of tagged and differentially labelled peptide samples therefore determines the relative quantities as well as the sequence identities of the components of protein mixtures in a single, automated operation.

In a fifth aspect of the invention, there is provided a use of a capture reagent in accordance with the first aspect in analysis of proteomes (or parts of the proteome) from two or more different samples.

In another aspect of the invention, there is provided a use of a capture reagent in accordance with the first aspect in separation of two or more peptides.

In a sixth aspect of the invention there is provided a kit for use in the analysis of proteomes, said kit comprising a capture reagent in accordance with the first aspect.

Preferably, such a kit comprises at least two individually identifiable capture reagents. For example, one with hydrogens present in the linker and one with deuteriums, such that the kit can be used to measure the relative quantities of proteins from at least two different samples.

Following chemical capture and separation, each proteome sample has the potential to create many hundreds of fractions of peptides requiring analysis with even more complexity being required if sequencing of each peptide is necessary. The workflow of different embodiments of the invention are shown in FIGS. 2 to 4. Where the reporter molecule is a fluorescent moiety, the proteolysis step is removed and purification and analysis would normally be performed by 2D-gel electrophoresis. For solution phase labelling of peptides a soluble macromolecule carrier is used (FIG. 4).

For example, where isotopically labelled chemical capture reagents are used, the repetitive steps that are involved in large scale sample analysis include the collection of isotope-labelled peptides from the chromatography column, spotting these onto the appropriate target depending on the choice of mass spectrometer (e.g. the MALDI target together with matrix where this is the mass spectrometer of choice) and analysis of each fraction. Automation of this part of the process would increase both the throughput and accuracy of the collected data.

Accordingly in a seventh aspect of the invention there is provided an automated system for analysis of proteomes. For mass spectrometric detection of isotopically labelled samples, a suitable automated system may comprise use of a capture reagent in accordance with the first aspect of the invention, automated steps of BHLC and an in-line fraction collector and spotter.

Strategies for automation in the case of MALDI MS could, for example, involve collection of the samples from the chromatographic separation in a microtitre plate, or reducing the flow rate using microbore HPLC. Alternatively, where electrospray is the analytical method of choice, thermo ion spray could be used to concentrate the sample as it exits the column.

### BRIEF DESCRIPTION OF THE DRAWINGS

For the purposes of clarity, certain embodiments of the present invention will now be described by way of example with reference to the following figures:

FIG. 1 shows a reaction scheme for preparation of a compound of Formula II;

FIG. 2 shows a reaction scheme for relative peptide quantification by chemical capture of proteins followed by proteolytic digestion;

FIG. 3 shows a reaction scheme for relative peptide quantification by chemical capture of peptides following proteolytic digestion of protein samples;

FIG. 4 shows a reaction scheme for solution phase labelling of proteins with a soluble carrier;

FIG. 5 shows a reaction scheme for the preparation of Maleimidocaproic acid-Sieber-CPG functionalised solid support;

FIG. 6 shows a reaction scheme for the preparation of Maleimidocaproamide-Rink-CPG

FIG. 7 shows a reaction scheme for the preparation of Maleimidocaproamide-Indole-Aminomethylated Polystyrene

FIG. 8 shows an HPLC trace of 6-Maleimidocaproamide (hard line denoting absorption at 215 nm, dotted line denoting absorption at 256 nm);

FIG. 9 shows a calibration curve of HPLC integration against mass of Maleimidocaproamide;

FIG. 10 illustrates the kinetics of cleavage of Maleimidocaproamide acid from Maleimidocaproamide-Sieber-CPG;

FIG. 11 shows an HPLC trace for a Creatine Kinase-Trypsin digest (hard line denoting absorption at 215 nm, dotted line denoting absorption at 256 nm);

FIG. 12 shows a MALDI mass spectrum of chemically captured and cleaved Creatine Kinase on Maleimidobutyramide-Sieber-CPG;

FIG. 13 depicts the normalised cleavage of Maleimidobutyramide from Maleimidobutyramide-Sieber-CPG;

FIG. 14 shows a calibration of H₆ and D₆ Maleimidobutyric acid and concentration;

FIG. 15 shows a MALDI mass spectrum of chemically captured and cleaved Glyceraldehyde 3-Phosphate Dehydrogenase on Maleimidobutyramide-Sieber-CPG;

FIG. 16 shows a mass spectrum depicting 1:4 (H:D) capture of Laminin Peptide 925-933 on H₆ and D₆ Maleimidobutyramide-Sieber-CPG (shown with sodium adduct);

FIG. 17 shows a MALDI mass spectrum depicting 1:1 (H:D) capture of Laminin Peptide 925-933 on H₆ and D₆ Maleimidobutyramide-Sieber-CPG (shown with sodium adduct);

FIG. 18 shows a MALDI mass spectrum depicting 4:1 (H:D) capture of Laminin Peptide 925-933 on H₆ and D₆ Maleimidobutyramide-Sieber-CPG (shown with sodium adduct);

FIG. 19 shows a comparison of experimentally derived and the theoretical ratio of D₆:H₆-Maleimidobutyramide-Laminin;

FIG. 20 shows a MALDI mass spectrum of chemically captured and cleaved [Val-OH]—α-Melanocyte Peptide on NHS-Adipic acid-Sieber-CPG; and

FIG. 21 shows a MALDI mass spectrum of chemically captured and cleaved Bag Cell Peptide 1-8 on NHS-Adipic acid-Sieber-CPG.

### DETAILED DESCRIPTION OF THE INVENTION

### Abbreviations

In the interests of brevity, the following abbreviations are used throughout the description:

| | |
|---|---|
| CHAPS | 3-[(3-Cholamidopropyl)dimethylammonio]-1-propanesulfonate |
| CPG | Controlled pore glass |
| DCM | Dichloromethane |
| DMF | N,N-dimethylformamide |
| DMSO | Dimethylsulfoxide |
| EDAC | 1-ethyl-3-(3-dimethylaminopropyl)carbodiimide hydrochloride |
| Et₃N | Triethylamine |
| Fmoc | (9-Fluorenylmethoxycarbonyloxy) |
| HOBt | 1-hydroxybenzotriazole |
| MalBut | Maleimidobutyramide |
| MalCap | Maleimidocaproamide |
| NHS- | N-hydroxysuccinimidyladipamide |
| Adipic | |
| PEG | Poly(ethyleneglycol) |
| TBTU | O-(1H-benzotriazol-1-yl)-N,N,N′,N′-tetramethyluronium |
| | tetrafluoroborate |
| TCEP | Tris(2-carboxyethyl)phosphine hydrochloride |
| TFA | Trifluoroacetic acid |
| TRIS | Tris(hydroxymethyl)aminomethane |
| TSTU | O-(N-succinimidyl)-N,N,N′,N′-tetramethyluronium |
| | tetrafluoroborate |

### Reagents and Equipment

The long-chained amine controlled-pore glass (LCA-CPG) was purchased from CPG Inc, New Jersey, USA. D₆-aminobutyric acid was purchased from CDN Isotopes, Quebec, Canada Fmoc-Rink-polystyrene and 3-formyl-indolyl-acetomidomethyl polystyrene were purchased from CN Biosciences (UK) Ltd., Nottingham, England. The peptide Ac-Glu-Glu-Val-Val-Ala-Cys-AMC peptide was obtained from Bachem, Merseyside, UK. CombiZorb S-monoamine was purchased from Crawford Scientific, Strathaven. α-cyano-4-hydrocinnamic acid, CHAPS, urea, Ficoll PM70 and PD-10 desalting columns were obtained from Amersham Biosciences, Little Chalfont, England. Tris-(2-carboxyethyl) phosphine, hydrochloride was bought from Molecular Probes, AA Leiden, Netherlands. Sequencing grade trypsin was purchased from Promega, Southampton and Ziptips® from Millipore (UK) Ltd, Watford. Dimethylformamide, methanol, acetonitrile, dichloromethane and ether were all obtained from BDH, Poole. All other materials were purchased from Sigma-Aldrich Company Ltd., Dorset, England.

The Sieber linker was prepared according to the procedure published by Albericio et al (‘Preparation and Applications of Xanthenylamide (XAL) Handles for Solid-Phase Synthesis of C-Terminal Peptide Amides under Particularly Mild Conditions’, J. Org. Chem. (1996) 61, 6326-6339).

The D₀-maleimidobutyric acid and D₆-maleimidobutyric acid were synthesised according to the procedure by Rainer and Grahe (‘Method for the preparation of N-(carboxyalkyl)maleimides’, R. B. Rainer and G. F. Grahe, EP0847991).

The active substitution level (typical values ranged from 1-10 μmol) of the Fmoc on the carrier was calculated from the UV/VIS absorption spectra at 278 nm. The standard procedure was as follows: 2.0 mg of the prepared Fmoc solid support was accurately weighed out and suspended in 1.81 g of DMF:piperidine (1:1 w/w) solution. The reaction was allowed to proceed to 45 minutes whereby 100 μl of the supernatant was transferred to a 1 ml volumetric flask and diluted with DMF:piperidine (1:1 w/w) solution. The absorbance was measured at 278 nm and the loading calculated from the equation below.

[c(mmol/g)=(Abs(absorbance)×10⁴)/10,417(ε)]

HPLC was performed on a Gilson HPLC equipped with Gilson 234 Autoinjector, 321 pump, 170 diode array detector and FC203B fraction collector. The column used (unless otherwise specified) was a Pharmacia Sephasil ST 4.6/250 C18 reversed phase column. Solvent A was 0.1% trifluoroacetic acid/water, solvent B: 0.1% trifluoroacetic acid 90% acetonitrile/10% water. The gradient employed (unless otherwise stated) was 100% aqueous to 20% aqueous over 30 minutes, then 0% aqueous over five minutes, holding at 0% for 1 minute then increasing to 100% aqueous over the next five minutes.

NMR spectra were performed on the jeol JNM-LA300 FT NMR system in a suitable deuterated solvent.

Matrix assisted laser desorption ionisation mass spectroscopy (MALDI MS) was performed on a Bruker Biflex III instrument. Typically, 5 μl of the matrix (α-cyano-4-hydroxycinnamic acid at 10 mg/ml in 50:50 ethanol: acetonitrile) was diluted with 5 μl of the sample and 0.5 μl spotted onto a MALDI anchor target and a MALDI spectra recorded. The machine was calibrated either with [Glu]fibrinopeptide, apamin and angiotensin II or Bradykinin 1-7, Angiotensin I, angiotensin II, substance P, bombesin and adrenocorticotropic hormone 1-17.

Solvent from HPLC fractions were removed on a Savant 352 Speed vac concentrator with inline Savant RT490 refrigerated condensation trap and Edwards E2M5 high vacuum pump.

Electrospray MS and MS/MS analysis were performed on a Micromass Q-T of Ultima API tandem mass spectrometer fitted with a Z-spray nanoflow electrospray ion source. The mass spectrometer was operated in positive ion mode with a source temperature of 80° C., a counter current gas flow rate of 40 L/hr and with a potential of 3500V applied to the standard flow probe. The instrument was calibrated with a multipoint calibration using selected ions from the collision-induced dissociation of Glu-fibrinopeptide-B.

### Specific Examples

Synthesis of Immobilised Reagent

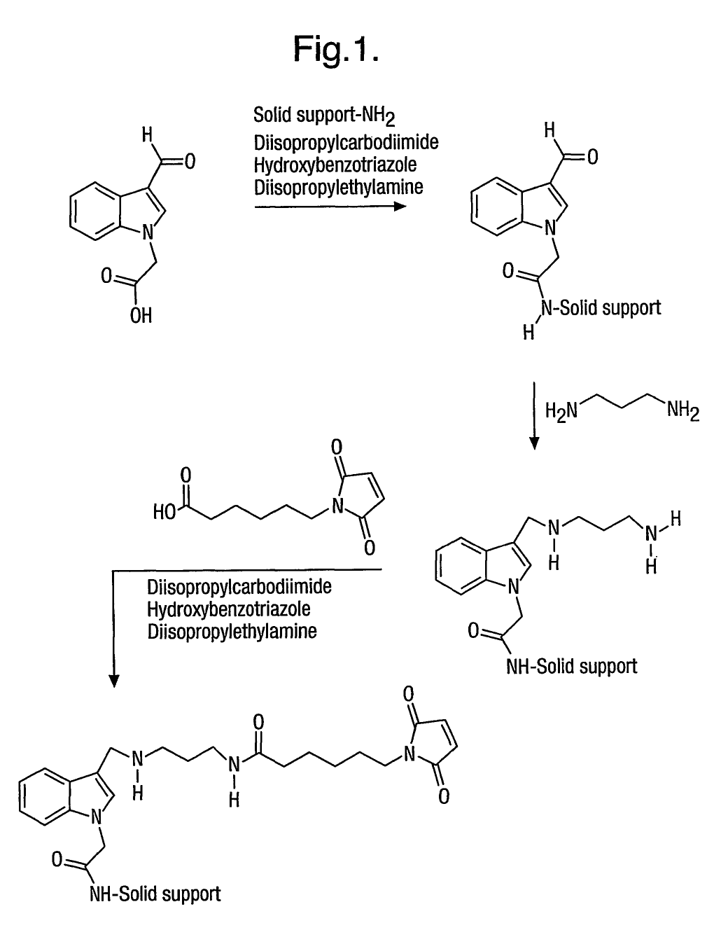

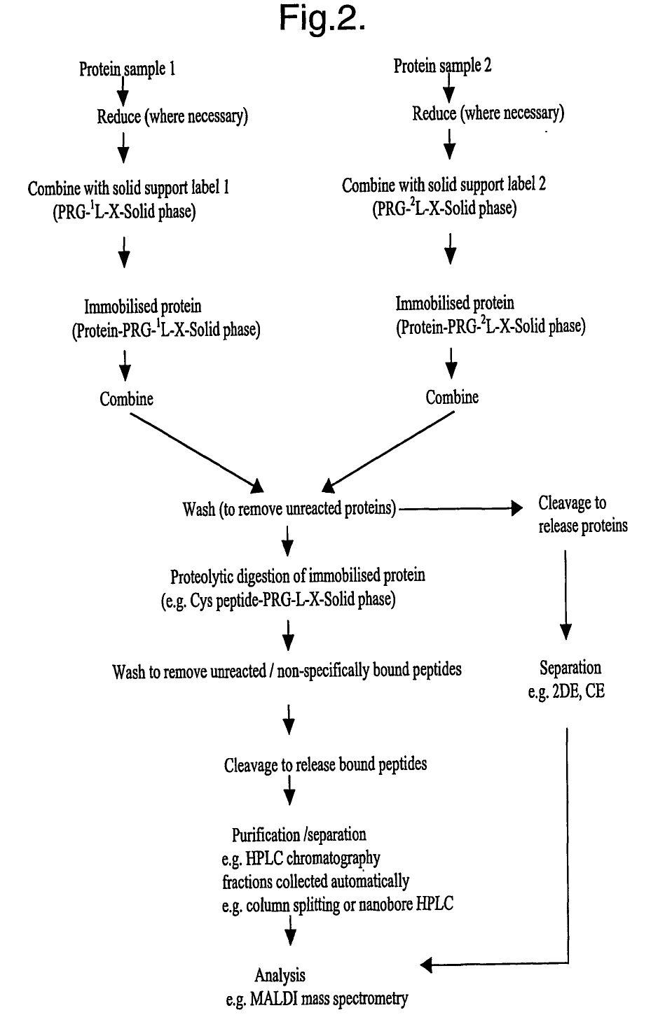

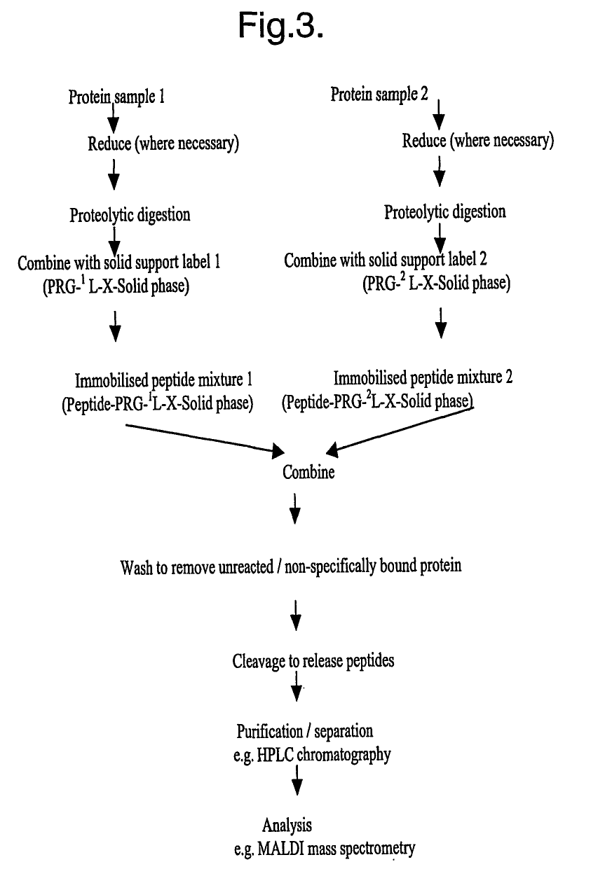

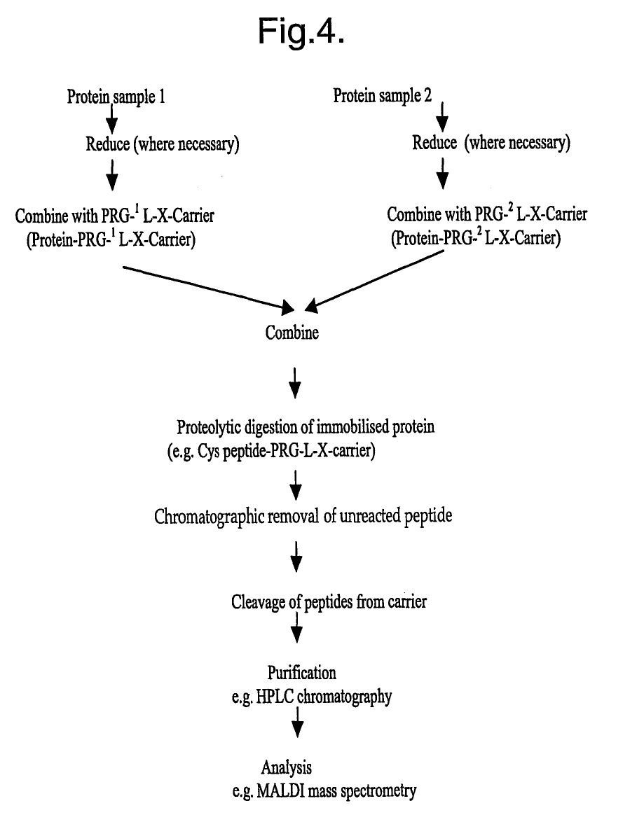

The synthesis of a cleavable linker on a solid support with a maleimide protein reactive group is achieved as shown in FIG. 1.

To the CombiZorb S-monoamine solid support (500 mg, 750 μmol/g) was added dichloromethane (10 ml) and dimethylformamide (1.5 ml). 3-Formyl-indol-1-yl-acetic acid (0.15 g, 0.75 mmol), N-hydroxybenzotriazole (0.40 g, 3.0 mmol), 1,3 diisopropylcarbodiimide (0.38 g, 3.0 mmol) and N,N-diisopropylethylamine (0.15 g, 1.1 mmol) were added and the reaction stirred for 3 days. The solid support was washed with water (2×10 ml), methanol (2×10 ml), DMF (1×10 ml) then 50:50 methanol:water (1×10 ml) and finally methanol (1×10 ml) and dried under a stream of air.

To the indole derivatised solid support as above (50 mg, indole substitution: 206 μmol/g, 10.3 μmol) was added dichloromethane (1 ml), 1,3-diaminopropane (188.8 μmol, 29.1 mg) and stirred at room temperature for 10 minutes. Sodium triacetoxyborohydride (188.8 μmol, 40.02 mg) was then added and the reaction stirred at room temperature for 9 hours.

To maleimidocaproic acid (42.2 mg, 200 μmol.) was added dichloromethane (2 ml) and DMF (300 μl) followed by N-hydroxybenzotriazole (0.108 g, 800 μmol), N,N-diisopropylethylamine (0.103 g, 800 μmol) and 1,3-diisopropylcarbodiimide (0.101 g, 125.3 μl, 800 μmol) and left at room temperature for 30 minutes. The derivatised indole solid support prepared above (40 mg) was then added and the reaction left roller mixing at room temperature (25° C.) for 17 hours.

The solid support was washed with water (2×2 ml), methanol (2×2 ml), water:methanol 50:50 (3×2 ml), water (2×2 ml) and finally methanol (2×2 ml) and dried under a stream of air.

Synthesis of Deuterated Chemical Capture Reagent

The incorporation of deuterium into the linker for simultaneous differential analysis of one or more samples is achieved using the same synthetic route as described above for the ‘hydrogen’ (i.e. non-labelled) species. The label is incorporated into the linker using the same reductive amination procedure used for incorporating the non-labelled linker. The deuterated linker has been represented in Formula II as the d4-1,3-diaminopropane derivative. There are a number of commercially available alternatives for this linker (CDN Isotopes).

| Structure | Chemical name | No of D's |
|---|---|---|
| NH₂CD₂CD₂CD₂CD₂NH₂ | 1,4-diamino-d8-diamine | 8 |
| NH₂CD₂CH₂CD₂NH₂ | 1,3-diaminodiamine-1,1-3,3-d4 | 4 |
| NH₂CH₂CD₂CH₂NH₂ | 1,3-propanediamine-2,2-d2 | 2 |

Fmoc-Sieber-CPG

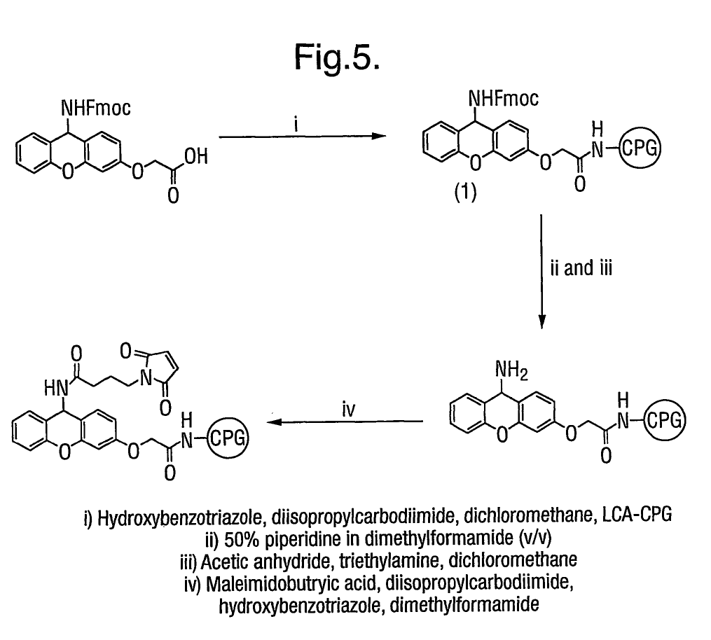

The LCA-CPG (500 mg, 19.25 μmol), Fmoc-Sieber linker (95 mg, 192.5 mol), diisopropylcarbodiimide (25 μl, 192.5 μmol) and hydroxybenzotriazole (26 mg, 192.5 μmol) were mixed together in dichloromethane and the reaction allowed to proceed for 15 hours. The solid support was then successively washed with dimethylformamide (2×10 ml), dichloromethane (2×10 ml) and diethyl ether (2×5 ml) before vacuum drying (FIG. 5).

Sieber-CPG

The Fmoc-Sieber-CPG (275 mg) was suspended in anhydrous dichloromethane (1 ml) and acetic anhydride (100 μl, 9.56 mmol) and triethylamine (100 μl) were added and the reaction allowed to mix for 1 hour at 25° C. The solid support was then collected by filtration and washed successively with dimethylformamide (2×2 ml), dichloromethane (2×4 ml) and diethyl ether (2×2 ml) before vacuum drying. The Fmoc group was removed from the resin by addition of 50:50 DMF:piperidine to the solid support for one hour followed by washing with dimethylformamide (2×2 ml), dichloromethane (2×4 ml) and diethyl ether (2×2 ml) before vacuum drying (FIG. 5).

Maleimidocaproic acid-Sieber-CPG

The Sieber-CPG (100 mg, 3.83 μmol (assume a loading of 38.3 mol/g)) was suspended in anhydrous dimethylformamide (500 μl) and maleimidocaproic acid (8 mg, 38.3 μmol), diisopropylcarbodiimide (6 μl, 38.3 μmol) and hydroxybenzotriazole (6 mg, 38.3 μmol) were added and the reaction mixed at 25° C. for 16 hours. The solid support was then collected by filtration and washed successively with dimethylformamide (2×2 ml), dichloromethane (2×2 ml) and diethyl ether (2×2 ml) before vacuum drying (FIG. 5).

Synthesis of Maleimidocaproamide-Rink-CPG

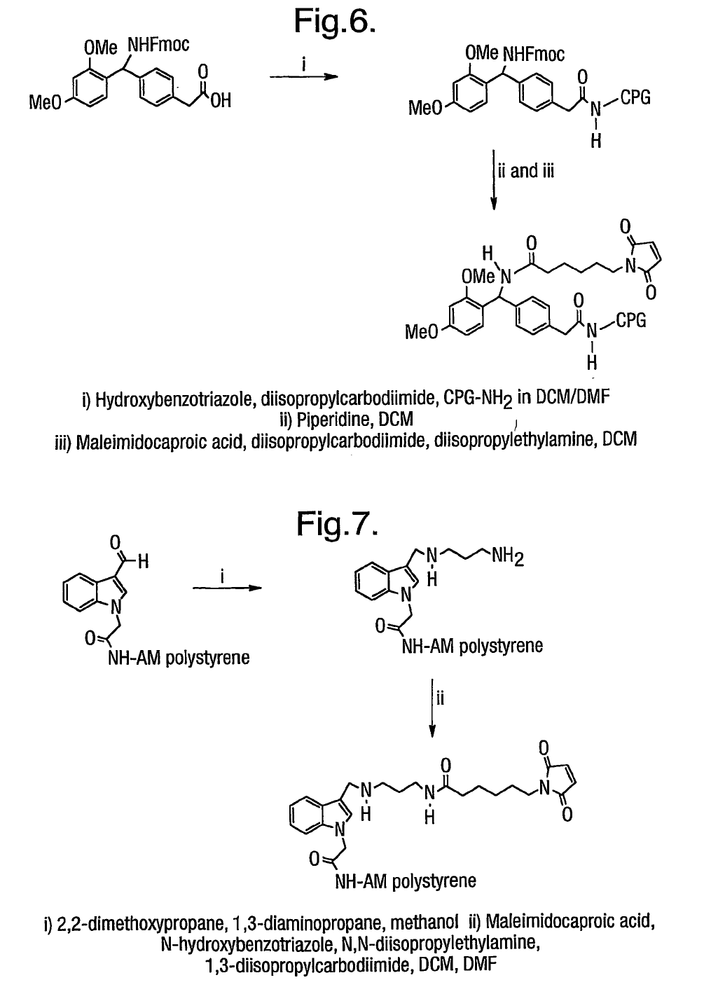

Maleimidocaproamide-Rink-CPG was synthesised as shown in FIG. 6. To the p-[(R,S)-a-[1-(9H-Fluoren-9-yl)-methoxyformamido]-2,4-dimethoxybenzyl]-phenoxyacetic acid (Fmoc Rink) linker (50 mg) was added dichloromethane (1 ml), hydroxybenzotriazole (12.5 mg), DMF (70 μl) and warmed to dissolve. Diisopropylcarbodiimide (14.5 μl) was then added followed by the LCA-CPG (50 mg, 38.3 μmol/g) and the mixture roller-mixed at 25° C. for 3 hours. The supernatant was removed in vacuo and the resin washed with dichloromethane (2×2 ml), methanol:water 50:50 (1×2 ml) and finally methanol (2×2 ml) and dried in a stream of air (51.1 mg). Then dichloromethane (0.8 ml) was added followed by piperidine (200 μl) and left for 30 minutes at 25° C. The sample was washed and dried as above to give a white solid (35 mg).

To maleimidocaproic acid (25 mg) was added diisopropylcarbodiimide (18.5 μl) and dichloromethane (0.5 ml) followed by diisopropylethylamine (20 μl) and Rink-CPG (total, as above). The mixture was roller-mixed for 2 hours at 25° C. and then washed and dried as above. To maleimidocaproamide-Rink-CPG (4.5 mg) was added sodium carbonate buffer (pH 8.0, 1 ml) followed by laminin fragment 925-933 (0.1 mg) and left for 16 hours at 25° C. The supernatant was removed and the solid support washed with water (500 μl). Then 10% TFA in water (50 μl) was added, the sample zip tipped after 60 minutes and a MALDI spectra was recorded.

Synthesis of Maleimidocaproamide-Indole-Aminomethylated polystyrene

To (3-formylindolyl)acetamidomethyl polystyrene solid support (200 mg) was added 2,2-dimethoxypropane (400 μl) and 1,3-diaminopropane (80 μl) followed by methanol (2 ml) and left overnight. Sodium triacetoxyborohydride (0.208 g) was added to half of the sample and the reaction roller mixed at 25° C. for 24 hours. The solid support was washed with methanol (2×2 ml), water (2×2 ml), water:methanol (50:50, 2 ml), methanol (2×2 ml) and dried under a stream of air.

To maleimidocaproic acid (84.4 mg) was added dichloromethane (4 ml) and DMF (600 μl) followed by N-hydroxybenzotriazole (0.216 g), N,N-diisopropylethylamine (0.206 g) and 1,3-diisopropylcarbodiimide (0.202 g) and left at room temperature for 30 minutes. The derivatised indole solid support (as above, 43 mg) was added to the activated maleimidocaproic acid (2.3 ml) and the reaction left at 25° C. for 17 hours. The solid support was washed with water (2×2 ml), methanol (2×2 ml), water:methanol 50:50 (3×2 ml), water (2×2 ml) and finally methanol (2×2 ml) and dried under a stream of air.

To the p-[(R,S)-a-[1-(9H-Fluoren-9-yl)-methoxyformamido]-2,4 diethoxybenzyl]-phenoxyacetic acid (Fmoc Rink) linker (50 mg) was added dichloromethane (1 ml), hydroxybenzotriazole (12.5 mg), DMF (70 μl) and warmed to dissolve. Diisopropylcarbodiimide (14.5 μl) was then added followed by the LCA-CPG (50 mg, 38.3 mmol/g) and the mixture roller-mixed at 25° C. for 3 hours. The supernatant was removed in vacuo and the resin washed with dichloromethane (2×2 ml), methanol:water 50:50 (1×2 ml) and finally methanol (2×2 ml) and dried in a stream of air (51.1 mg). Then dichloromethane (0.8 ml) was added followed by piperidine (200 μl) and left for 30 minutes at 25° C. The sample was washed and dried as above to give a white solid (35 mg).

Capture of Laminin Fragment 925-933 on Maleimidocaproamide-Rink-CPG

To maleimidocaproic acid (25 mg) was added diisopropylcarbodiimide (18.5 μl) and dichloromethane (0.5 ml) followed by diisopropylethylamine (20 μl) and Rink-CPG (total, as above). The mixture was roller-mixed for 2 hours at 25° C. and then washed and dried as above. To maleimidocaproamide-Rink-CPG (4.5 mg) was added sodium carbonate buffer (pH 8.0, 1 ml) followed by laminin fragment 925-933 (0.1 mg) and left for 16 hours at 25° C. The supernatant was removed and the solid support washed with water (500 μl). Then 10% TFA in water (50 μl) was added, the sample zip tipped after 60 minutes and a MALDI spectra was recorded. A peak at m/z 1177.4 was observed corresponding to laminin fragment 925-933 plus maleimidocaproamide.

Solid Phase Synthesis of 6-maleimidocaproamide

To Fmoc-Rink-polystyrene solid support (0.5 g, 345 μmol) was added dimethylformamide (3 ml) and piperidine (0.6 ml) and left roller mixing at 25° C. for 60 minutes. The solid support was then washed with dimethylformamide (2×5 ml), dichloromethane (2×5 ml), methanol (2×5 ml), then solvent allowed to evaporate under a stream of air.

To 6-maleimidocaproic acid (50 mg, 0.23 mmol) was added TBTU (76 mg, 0.25 mmol), dichloromethane (0.5 ml) and dimethylformamide (0.5 ml). After 30 minutes a further 1 ml dimethylformamide was added. The Rink-polystyrene solid support (as above) was then added and left roller mixing for 3 hours at 25° C. The solid support was washed with dichloromethane (2×3 ml), methanol (1×2 ml), water (2×3 ml), methanol:water 50:50 (1×3 ml), methanol (2×3 ml) and finally dried under a stream of air. To the maleimidocaproic acid-Rink-polystyrene (7.09 mg, 2.4 μmoles assuming 50% yield), was added dichloromethane (638 μl) and trifluoroacetic acid (63.8 μl) and left at 25° C.

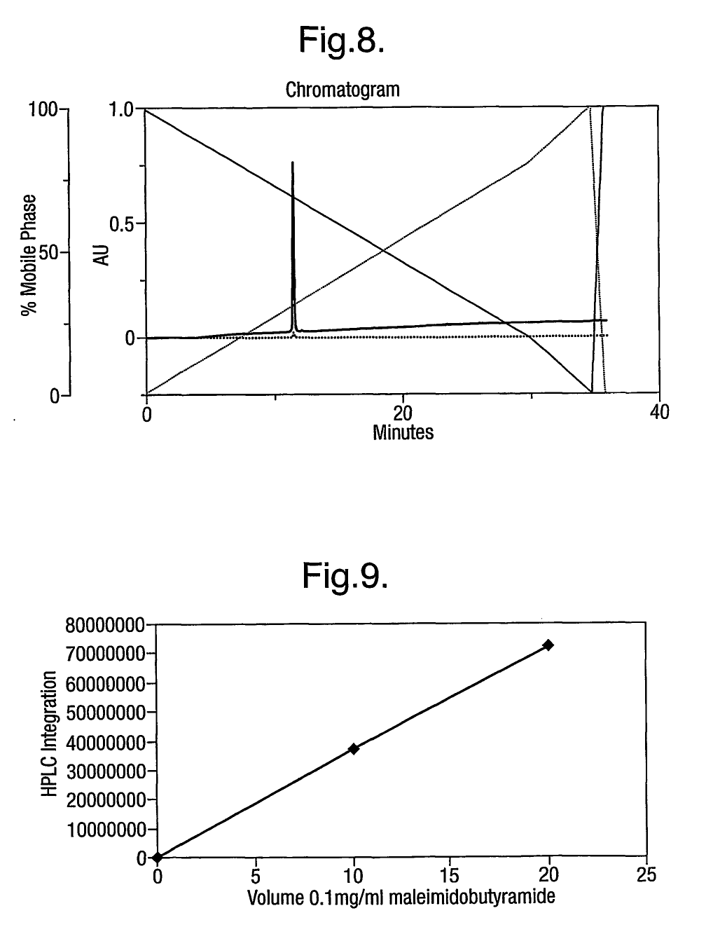

To the maleimidocaproic acid-Rink-CPG solid support was added dichloromethane (2 ml) followed by trifluoroacetic acid (20 μl) and left for 20 hours at 25° C. Then 5 μl was removed and evaporated and 0.1% trifluoroacetic acid/water added (1000 μl) of which 135 μl was injected onto a C18 column as above (FIG. 6). The solid support was then filtered from the solution using a glass wool plugged pasteur pipette and a further 5 ml of dichloromethane used to wash the resin. The solvent was evaporated in vacuo to give 6-maleimidocaproamide (4.5 mg) and then analysed by NMR δH (300 MHz, CDCl₃) 1.28 (2H, dd, CH₂), 1.50-1.66 (4H m 2×CH₂), 2.17 (2H t J=7.5 Hz, CH₂), 3.45 (2H t J=6.9 Hz CH₂), 5.54 (1H s N—H), 5.79 (1H s N—H), 6.67 (2H s H—C═C—H. The retention time of the maleimidocaproamide was determined to be 11.60 mins, the same retention time as observed for acidolytic cleavage of Maleimidocaproamide-Sieber-CPG (FIG. 8).

Calibration of HPLC Integration vs Mass of Maleimidocaproamide

To maleimidocaproamide (0.54 mg, 2.57 μmol) was added 0.1% trifluoroacetic acid/water (5.4 ml) and both 10 μl and 20 μl diluted to 135 μl with 0.1% trifluoroacetic acid/water and analysed by HPLC at 215 nm. An integration of 1.0×10⁹ was found to equate to 27.57 μg of maleimidocaproamide (FIG. 9).

Cleavage Kinetics of Maleimidocaproic acid-Sieber-CPG

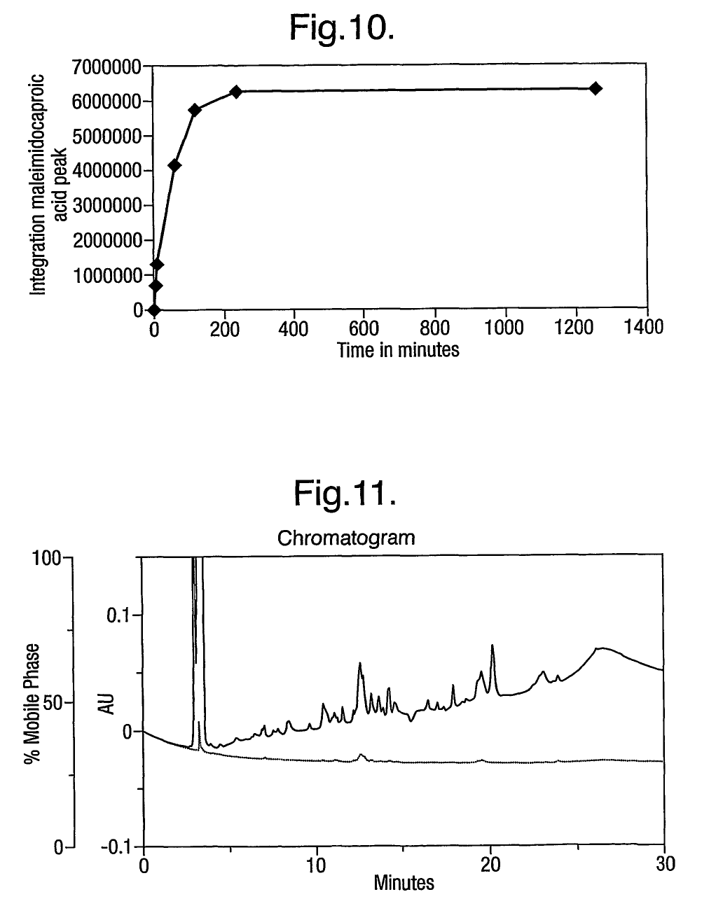

To maleimidocaproic acid-Sieber-CPG solid support (5.0 mg, 4.1 μmol/g) was added water (490 μl) and trifluoroacetic acid (5 μl) at 26° C. At specific time periods 20 μl was removed and diluted with water (115 μl). The solution was then injected onto the HPLC. The integration of the maleimidocaproamide peak was calculated at each time point, which was plotted against cleavage time to determine the rate of cleavage (FIG. 10).

Chemical Capture of Glyceraldehyde 3-Phosphate Dehydrogenase Followed by Proteolysis

To glyceraldehyde 3-phosphate dehydrogenase (8.6 mg, 240 nmol) was added 6M urea 0.05M TRIS pH 8.0 (1.72 ml) followed by TCEP (2 mg/ml in 6M urea 0.05M TRIS pH 8.0, 103.5 μl) and roller-mixed for 15 minutes at 25° C. Then maleimidocaproic acid-Sieber-CPG (9.8 mg, 37.2 nmol) was added and roller mixed for five hours at 25° C. The supernatant was removed and the resultant solid support was washed with water (2×1 ml), 6M urea, 0.05M TRIS/pH 8.0 (2×1 ml) and finally water (2×1 ml).

To the above protein-bound solid support was added trypsin (20 μg/ml, 25 μl) and 50 mM carbonate pH 8.0 (0.5 ml) and roller mixed for 1 hour at 25° C. The solid support was then warmed to 37° C. for 20 minutes and then the supernatant discarded. The solid support was washed with methanol (200 μl) then dichloromethane (200 μl) and finally 200 μl of dichloromethane in 1% trifluoroacetic acid was added and left 18 hours. The supernatant was removed and both the supernatant and solid support were allowed to evaporate. 5% trifluoroacetic acid in water (100 μl) was then added and allowed to react at 25° C. for 3 hours. The supernatant was then added to the dried down dichloromethane/trifluoroacetic acid fraction.

A Ziptip® was equilibrated in 0.1% trifluoroacetic acid/water and 10 μl of the cleaved Sieber-CPG adsorbed onto it, debinding in 50% acetonitrile/50% water with 0.1% trifluoroacetic acid. MALDI MS was performed on the material. As expected, two peaks were observed with m/z ratios of 1709.87 and 2080.18 corresponding to sequence 232-245 plus maleimido caproamide and 232-248 plus maleimido caproamide, respectively.

Proteolysis of Creatine Phosphokinase Without Chemical Capture

To creatine phosphokinase (2.0 mg, 47 nmol) was added 100 mM carbonate buffer pH 8.0 (1.00 ml) followed by trypsin (35 μg, 20 μg/ml) and left roller mixing at 25° C. for 16 hours. Then TCEP (2 mg/ml, 119 μl, 830 nmol) was added and the reaction left for a further 30 minutes at 25° C. 20 μl was removed and added to 5% trifluoroacetic acid/water (115 μl) and HPLC performed collecting one minute fractions, between 6 and 22 minutes (FIG. 11). Each 1 ml fraction was evaporated in vacuo and dissolved in 5 μl of 0.1% trifluoroacetic acid/water and a MALDI MS spectra measured. The following peaks were observed, together with assignments (Table 1).

TABLE 1
m/z Ratios for Digestion of Creatine Phosphokinase
(I and II represent two polypeptide chains)

| Fraction | Peak number | | | | | | | | |
|---|---|---|---|---|---|---|---|---|---|
| number | 1 | 2 | 3 | 4 | 5 | 6 | 7 | 8 | 9 |
| 5 | 914.4 | 936.4 | 952.4 | 986.4 | 1008.5 | | | | |
| | | | | (97-105 II) | (97-105 + | | | | |
| | | | | | Na⁺ II) | | | | |
| 7 | 759.2 | 983.1 | 1130.2 | 1152.2 | | | | | |
| | (210-215 I) | (216-223 I) | (139-148 | (139-148 + | | | | | |
| | | | II) | Na⁺ II) | | | | | |
| 8 | 759.1 | 1037.2 | 1130.2 | 1150.3 | 1193.3 | 1458.3 | 1714.4 | | |
| | (210-215 I) | (243-251 | (139-148 | | (237-247 I) | (139-151 I) | (12-25 II) | | |
| | | II) | II) | | | | | | |
| 9 | 816.1 | 907.2 | 950.2 | 970.2 | 986.2 | 1130.1 | 1530.2 | 2595.6 | 2861.2 |
| | | (308-314 | (253-259 + | (33-40 + | (97-105 II) | (139-148 | (117-130 | (108-130 | (342-366 I) |
| | | II) | Na⁺ II) | Na⁺ II) | | II) | II) | II) | |
| 10 | 1269.2 | 2861.1 | | | | | | | |
| | (305-314 | (342-366 I) | | | | | | | |
| | II) | | | | | | | | |
| 11 | 1269.3 | 2008.4 | | | | | | | |
| | (305-314 | (321-341 | | | | | | | |
| | II) | II) | | | | | | | |
| 13 | 1507.2 | 1529.2 | | | | | | | |
| | (157-170 | (157-170 + | | | | | | | |
| | II) | Na⁺ II) | | | | | | | |
| 14 | 1643.1 | 1659.2 | 1675.1 | 2870.6 | 3775.7 | | | | |
| | (224-236 | | (224-236 + | (267-292 | (93-130 I) | | | | |
| | II) | | Na⁺ II) | II) | | | | | |
| 15 | 1643.0 | 1659.1 | 1675.0 | 3604.7 | 3645.5 | | | | |
| | (224-236 | | | | (178-209 | | | | |
| | II) | | | | II) | | | | |

Chemical Capture of Creatine Phosphokinase

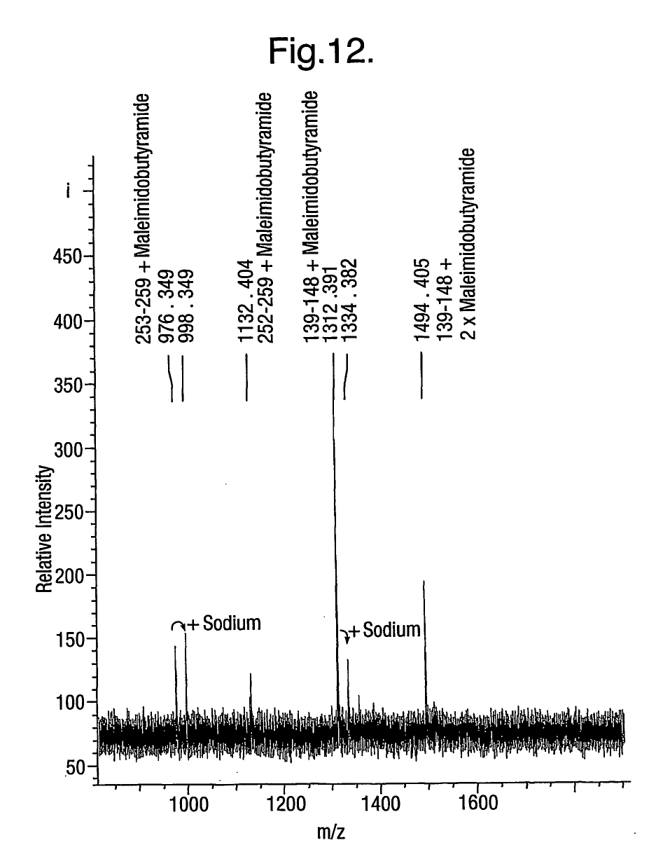

To creatine phosphokinase (2.93 mg) was added 0.1 M phosphate pH 8.0 (1.47 ml) and 0.5 ml removed. Trypsin (100 μl, 0.1 mg/ml in 50 mM acetic acid) was then added and incubated for 30 minutes at 37° C. Then TCEP (14.7 μl, 2 mg/ml in 0.1 M phosphate pH 8.0) was added and left at 37° C. for a further 2 hours. 93 μl of this solution was removed and added to MalBut-Sieber-CPG (5.72 mg) and left for 90 minutes at 25° C. The supernatant was removed and water (500 μl) added to the solid support, mixed by multiple inversion and then the supernatant discarded. A solution of 8M urea, 4% CHAPS, 40 mM Tris pH 8.0 (500 μl) was added and roller mixed for 10 minutes before discarding the supernatant. Then water (500 μl) was added, mixed by multiple inversion and the supernatant discarded, followed by addition of 0.1 M phosphate pH 8.0, 3 M sodium chloride (500 μl) and roller mixed for 60 minutes. The solid support was washed twice more with water (500 μl), mixing by multiple inversion and the supernatant discarded. Finally 1% trifluoroacetic acid in water (50 μl) was added and left overnight (16 hours) at room temperature (25° C.) and a MALDI spectrum recorded (FIG. 12).

Maleimidobutyric acid-Sieber-CPG (X D₀-MalBut, Y D₆MalBut)

The Sieber-CPG (100 mg, 3.83 μmol) was suspended in anhydrous dimethylformamide (500 μl) and the D₀- or D₆-maleimidobutyric acid (7 mg, 38.3 μmol), diisopropylcarbodiimide (6 μl, 38.3 μmol) and hydroxybenzotriazole (6 mg, 38.3 μmol) were added and the reaction mixed at 25° C. for 16 hours. The resin was then collected by filtration and washed successively with dimethylformamide (2×2 ml), dichloromethane (2×2 ml) and diethyl ether (2×2 ml) before vacuum drying.

Cleavage Reactions

In a typical test cleavage reaction, 2 mg of the prepared Mal-Sieber-CPG resin was accurately weighed into an Eppendorf tube and 5% aq. TFA added. The reaction was mixed for 2 hours and then 2041 removed and diluted to 135 μl with 0.1% aqueous TFA and an HPLC performed. The desired cleavage product was found to have a retention time of 8.0±0.2 minutes. The loading on the resin was then calculated from the area under the peak compared to known quantities of D₆ or D₀-maleimidobutyric acid standard from a dilution series.

Relative Cleavage Kinetics D₀ and D₆ Maleimidobutyric acid-Sieber-CPG

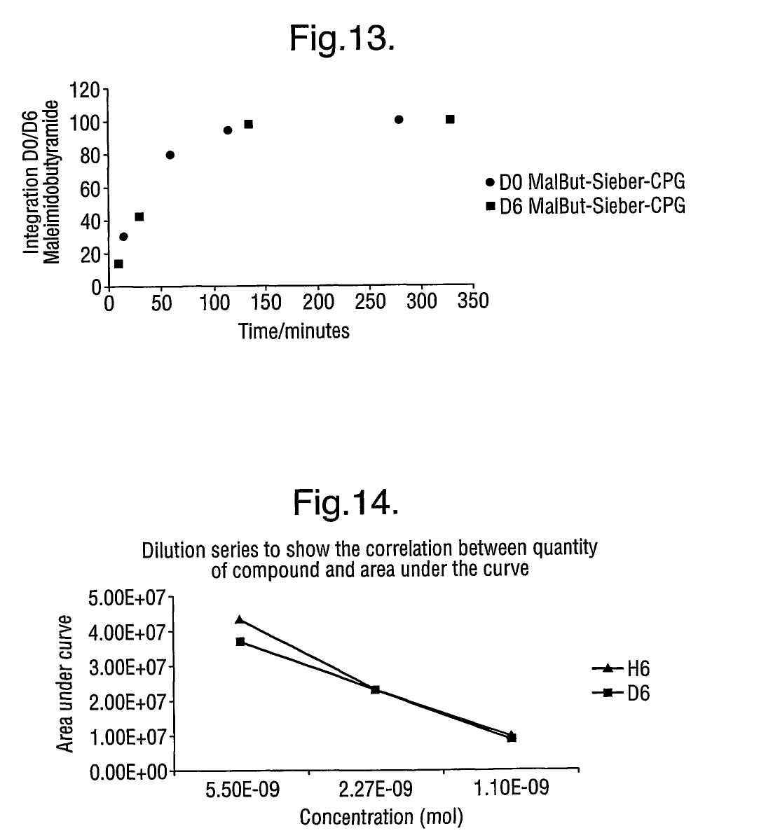

To the D₀ or D₆ MalBut-Sieber-CPG solid support (N mg) was added 1% trifluoroacetic acid in water (N×100 μl) at 25° C. At specific time periods 20 μl was removed and diluted with water (115 μl) and injected onto the HPLC. The integration of the maleimidobutyramide at 8 mins was calculated in order to establish the kinetics of the reaction (FIG. 13).

Determination of the Substitution of maleimidobutyric acid from the Area Under the HPLC Peak.

A series of dilutions of the D₀-maleimidobutyric acid and D₆-maleimidobutyric acid were performed to determine the loading of the resin after the cleavage reaction. Thus it was calculated that an area of 6.69×10⁶ equates to 1×10⁻⁹ moles of maleimidobutyric acid at 215 nm (FIG. 14).

Chemical Capture of Glyceraldehyde 3-phosphate Dehydrogenase (GAPDH) On Maleimidobutyric acid-Sieber-CPG Solid Support

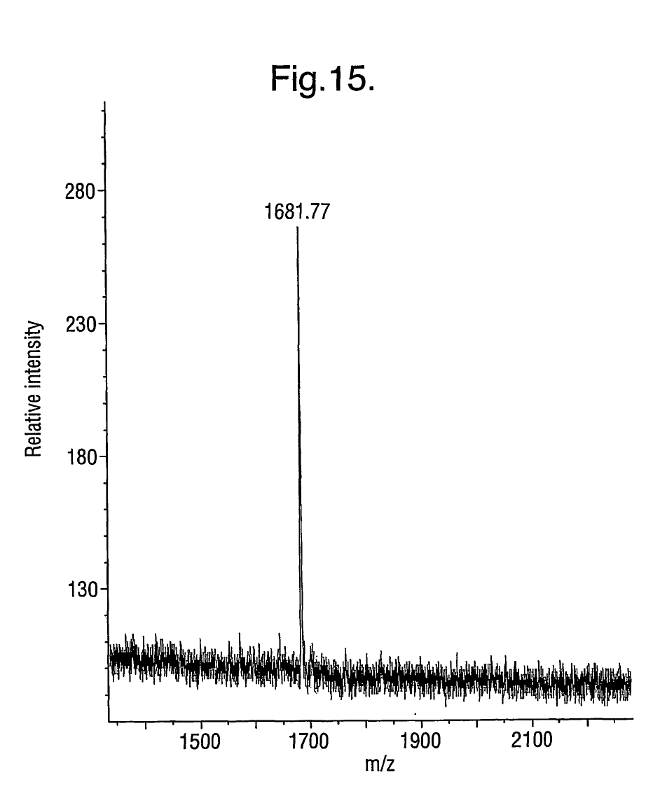

To GAPDH (1.33 mg, 37 nmol) was added lysis buffer (0.577 ml; 8M urea, 4% CHAPS (w/v), 40 mM tris pH 8.0) followed by TCEP (10 mM, 61 μl, 610 nmol) and incubated for 35 minutes at 37° C. MalBut-Sieber-CPG (17.8 mg) was added to the above reduced protein solution (55.3 μl) and left for 30 minutes at 37° C. The solid support was then washed with water (5×500 μl). Then N-acetyl cysteine (2 mg/ml in 0.1 M carbonate pH 8.0, 104.5 μl, 1.28 μmol) was added and left for 15 mins at 25° C. The solid support was washed with water (3×500 μl). 5.8 μl trypsin at 20 μg/ml in 0.1 M carbonate pH 8.0 was added to above solid support followed by 94.2 μl of 0.1M carbonate pH 8.0 and left at 25° C. for 16 hours. The solid support was then washed with water (4×500 μl), methanol:water 50:50 (2×500 μl), methanol (2×500 μl) and finally water (3×500 μl). 1% trifluoroacetic acid in water (100 μl) at 25° C. was then added and a MALDI MS recorded after 2 hours (FIG. 6). MS-MS analysis was performed by dilution of the peptide to a concentration of approximately 100 μfmol/μl followed by direct infusion into the mass spectrometer at a flow rate of 250 nl/min (FIG. 15).

The peptide was shown to have the amino acid sequence:

Val-Pro-Thr-Pro-Asn-Val-Ser-Val-Val-Asp-Leu-Thr-Cys(MalBut)-Arg corresponding to sequence 232-245 of rabbit GAPDH. The experimental results are in complete agreement with those in the literature for this sequence (VPTPNVSVVDLTCR; SwissProt accession number: P46406).

One Pot Tryptic Digestion and Capture of Glyceraldehyde 3-phosphate Dehydrogenase On Maleimidobutyric acid-Sieber-CPG

To GAPDH (1.15 mg) was added 0.1 M tris pH 8.0 (200 μl) followed by trypsin (40 μg/ml, 0.15 ml) together with H₆ MalBut-Sieber-CPG (5.0 mg) and TCEP (1 mg/ml in water, 23.5 μl). The reaction was left roller mixing at 25° C. for 16 hours then washed twice with water (1.5 ml), once with methanol:water 50:50 (1.5 ml), once with methanol (1.5 ml) and finally twice with water (1.5 ml). 1% TFA/water (100 μl) was then added and left for 2 hours at room temperature. A MALDI spectra was recorded. Peaks at m/z 1681.9 and mz/z 2052.2 were observed, corresponding to sequence 232-245 plus MalBut and 232-248 plus MalBut, together with an extra peak at m/z 1763.9 and 1828.2, the former being 307-320, the latter being unidentified.

Kinetics of Capture of Laminin Fragment 925-933 on Maleimidobutyric acid-Sieber-CPG

To the laminin fragment 925-933 in 0.1 M phosphate pH 8.0 (1 mg/ml, 50 μl) was added TCEP (2 mg/ml in water, 7.4 μl) and left for 60 minutes at 25° C. Then D₆ MalBut-Sieber-CPG (5.85 mg) was added to 29.5 μl of the above solution. At 60 minutes, 2 μl was removed and analysed by HPLC. No trace of the peptide fragment was observed.

Differential Analysis of Peptides on a D₀ and D₆ Maleimidobutyric acid-Sieber-CPG Solid Support

Three times 5.0 mg of the H₆ linker (5.87 nmol) were weighed out into 0.5 ml Eppendorf tubes together with the same weight of the D₆ linker into three separate tubes (3.1 μmol). Reduced laminin fragment 925-933 (0.2 mg/ml in 0.1M phosphate pH 8.0, 207 nmol) was added to the H₆ and D₆ linkers as follows:

| | Volume | | |
|---|---|---|---|
| | 0.2 mg/ml | | |
| | laminin | Volume 0.2 mg/ml | Volume 0.2 mg/ml |
| Solid support | fragment/μl | laminin fragment/μl | laminin fragment/μl |
| H₆ MalBut- | 1.9 (1) | 7.5 (2) | 7.5 (3) |
| Sieber-CPG | | | |
| D₆ MalBut- | 7.5 (4) | 7.5 (5) | 1.9 (6) |
| Sieber-CPG | | | |

Bold = tube number

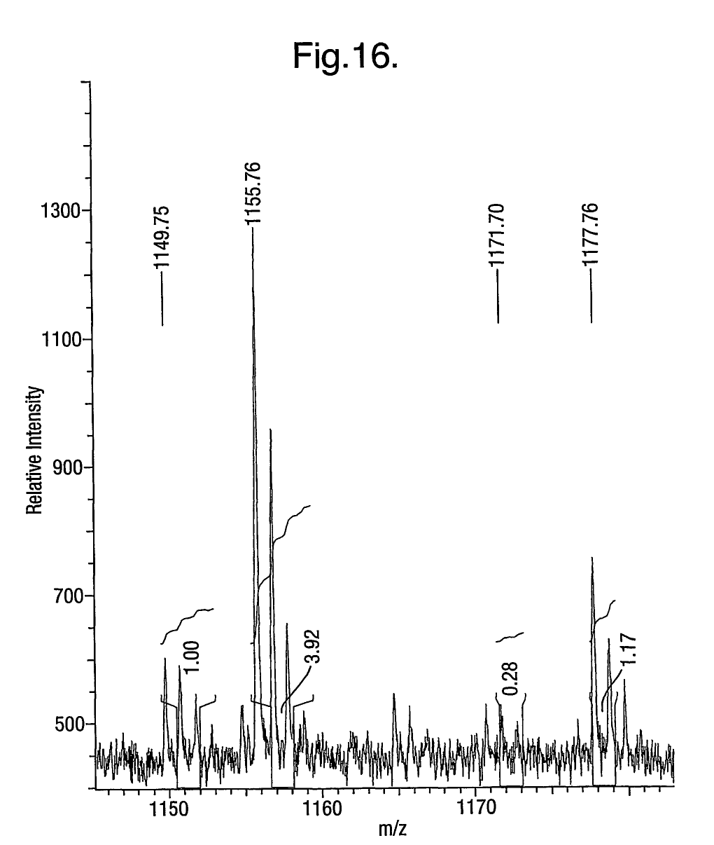

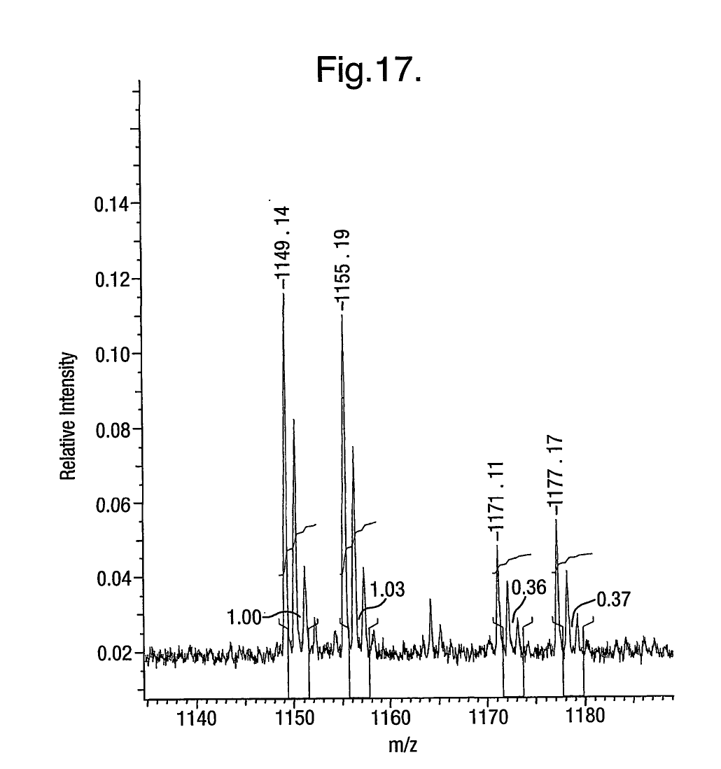

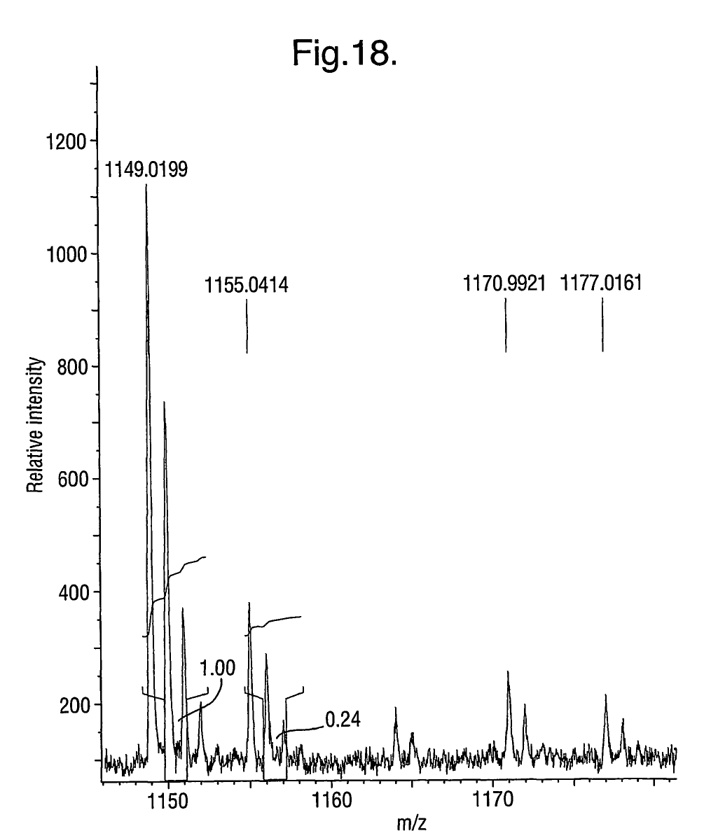

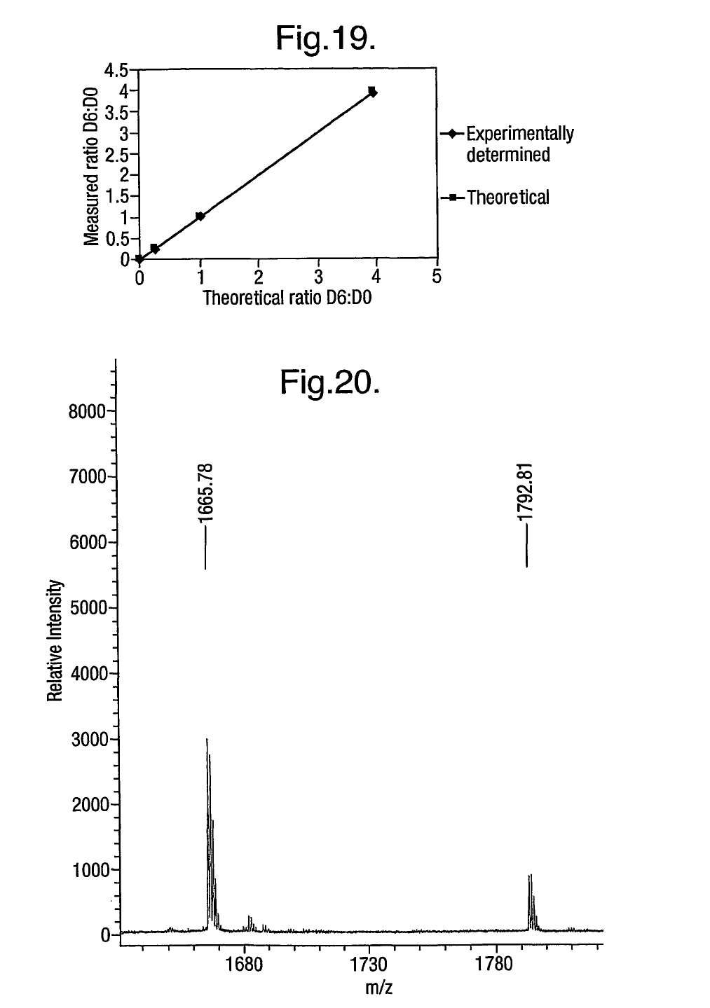

The total volume was then made up to 20 μl in all cases with 0.1M phosphate pH 8.0. The reactions were left at 25° C. for two hours with occasional agitation, then the supernatant removed. 400 μl of water was then added to the D₆ solid supports and the solid support transferred to the corresponding vial (4 to 1, 5 to 2 and 6 to 3). The particles were then thoroughly mixed by repeated agitation and the supernatant removed after centrifugation. 1% trifluoroacetic acid in water (100 μl) was then added to each of the three vials and left at 25° C. for 2 hours. A MALDI MS was recorded on the cleavage solution and the relative integrations of the laminin plus maleimidobutyramide peaks at m/z ratios of 1149 and 1155 were measured for each of the three (FIGS. 16-18). The measured relative integrations were 1:3.92, 1:1.03 and 1:0.24 compared with the expected values of 1:3.94, 1:1 and 1:0.25. As can be seen from FIG. 19 the theoretical and measured values overlay each other.

Lysine Reactive Solid Support Synthesis

The Fmoc group was removed from Fmoc-Sieber-CPG (60 mg, 2.30 μmol (assumed loading 38.31 μmol/g)) by addition of 50:50 DMF:piperidine (500 μl) to the solid support for one hour followed by washing with dimethylformamide (2×2 ml), dichloromethane (2×2 ml) and diethyl ether (2×1 ml) before vacuum drying.

The Sieber-CPG (50 mg, 1.92 mol) was suspended in anhydrous dimethylformamide (500 μl) and hexanedioic acid (7 mg, 38.3 μmol), diisopropylcarbodiimide (4 μt, 19.2 μmol) and hydroxybenzotriazole (4 mg, 19.2 μmol) were added and the reaction mixed at 25° C. for 16 hours. The resin was then collected by filtration and washed successively with dimethylformamide (2×2 ml), dichloromethane (2×2 ml) and diethyl ether (2×1 ml) before vacuum drying.

The resin was resuspended in anhydrous DMF (1 ml) and TSTU (5 mg, 19.2 μmol) and triethylamine (4 μl, 19.2 μmol) were added and the reaction mixed for 16 hours. The resin was then collected by filtration and washed successively with dimethylformamide (2×2 ml), dichloromethane (2×2 ml) and diethyl ether (2×1 ml) before vacuum drying.

Chemical Capture of γ-amino Group Functionality on NHS-Adipic acid-Sieber-CPG

[Val-OH]-α-melanocyte stimulating hormone (1 mg/ml in 0.1M phosphate pH 8.0, 20.5 μl, 12.3 nmol) was added to NHS-Adipic-Sieber-CPG (2.47 mg, 12.3 μmol assuming substitution=5 μmol/g) and left for 60 minutes at 25° C. The solid support was washed with water (3×500 μl). Trifluoroacetic acid (1% in water, 1001 μl) was added to the solid and left at 25° C. for 1 hour and a MALDI spectrum then recorded (FIG. 20). As exepcted, major peaks at m/z ratios of 1665.78 and 1792.81 were observed corresponding to the unlabelled and labelled peptide, respectively.

Chemical Capture of α-amino Group Functionality on NHS-Adipic acid-Sieber-CPG

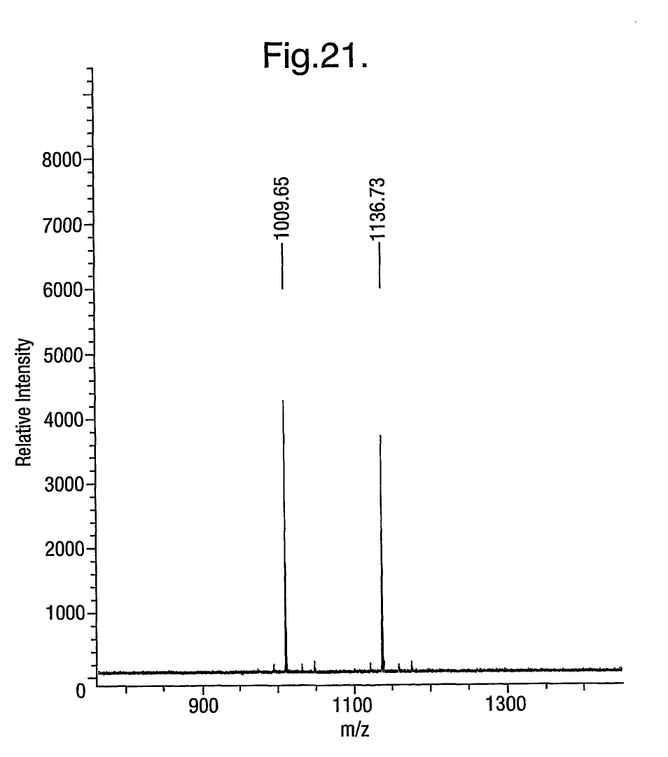

To bag cell peptide 1-8 (10.6 μl, 1 mg/ml in water, 10.5 nmol) was added 0.1M phosphate pH 8.0 (31.8 μl) and NHS-Adipic-Sieber-CPG (2.10 mg, 10.5 nmol assuming substitution=5 μmol/g) and left at 25° C. for 65 minutes. The solid support was washed with water (3×500 μl), 1% trifluoroacetic acid in water (100 μl) added and left at 25° C. for 60 minutes and then a MALDI spectrum recorded (FIG. 21). Major peaks at m/z ratios of 1009.65 (unlabelled peptide) and 1136.73 (labelled peptide) were observed, as expected.

Chemical Capture of Hydrophobic Peptides on Maleimidobutyric acid-Sieber-CPG

The potential for capture of hydrophobic peptides in organic solvents was investigated in dimethylsulphoxide and dimethylformamide:

(i) Dimethylformamide

To Ac-Glu-Glu-Val-Val-Ala-Cys-AMC (0.364 mg, 429 nmol) was added dimethylformamide (530 μl) and the peptide dissolved by heating to around 60° C. 15 μl of this solution was added to MalBut-Sieber-CPG (4.36 mg, 12.1 nmol) and left at 25° C. for 2 hours. The solid support was then washed twice with methanol (500 μl), and 1% TFA in methanol added (50 μl). The reaction was left overnight, then a MALDI spectrum recorded.

(ii) Dimethylsulphoxide

To Ac-Glu-Glu-Val-Val-Ala-Cys-AMC (0.14 mg, 165 nmol) was added dimethylsulphoxide (205 μl) and dissolved by heating to around 60° C. 18.5 μl of this solution was added to MalBut-Sieber-CPG (5.4 mg, 15.0 nmol) and left at 25° C. for 2 hours. The solid support was then washed once with dimethylsulphoxide (200 μl), once with methanol (500 μl) discarding the supernatant without roller mixing, then twice with methanol (500 μl), the methanol discarded and 1% TFA in methanol added (50 μl). The reaction was left overnight, then a MALDI spectrum recorded.

A peak at a m/z ratio of 1052.5 was observed in both cases corresponding to the expected Ac-Glu-Glu-Val-Val-Ala-Cys-AMC plus maleimidobutyramide.

Quantitative Capture of Reduced and Denatured Protein on Maleimidobutyric acid-Sieber-CPG Solid Support Followed by Proteolysis and Cleavage

To GAPDH (2.78 mg) was added lysis buffer (625 μl; 8M urea 4% CHAPS (w/v), 40 m phosphate pH 8.0) and TCEP (33 μl, 2 mg/ml in lysis buffer) and heated to 100° C. for 2 minute, then cooled rapidly under a stream of cold water. The volumes specified below were added to the H₆ and D₆ MalBut-Sieber-CPG solid supports and the mixture left roller mixing at 26° C. for 4 hours.

| | Mass solid | | | |
|---|---|---|---|---|
| | support/ | Identity solid | Volume | Volume lysis |
| Expt ID | mg | support | GAPDH/μl | buffer/μl |
| 1 | 5.15 | H₆ | 4 | 17 |
| 2 | 5.45 | H₆ | 16 | 5 |
| 3 | 5.23 | D₆ | 16 | 5 |
| 4 | 5.76 | D₆ | 4 | 17 |

Tube 1 was then transferred to tube 3, tube 2 transferred to 4 and each reaction washed twice with water (500 μl), roller mixing for 2 minutes then decanting off the supernatant. N-acetyl cysteine (10.4 μl, 10 mg/ml in 0.1 M phosphate pH 8.0) was added together with 0.1 M phosphate pH 8.0 (10 μl) and roller mixed at 26° C. for 10 minutes. The solid support was then washed with water (2×500 μl), roller mixing for 2 minutes prior to decanting the supernatant. Trypsin (40 μl, 100 μg/ml in 50 mM acetic acid) was added to each together with 0.1 M phosphate pH 8.0 (40 μl) and roller mixed at 26° C. for 30 minutes, then incubated at 37° C. in a water bath for 16 hours. The solid supports were washed with water (2×500 μl) and 8M urea, 4% (w/v) CHAPS, 40 mM phosphate pH 8.0 (500 μl) was added and roller mixed at 26° C. for 50 minutes. The solid support was washed once with water (500 μl) and then 0.1 M phosphate pH 8.0 3M sodium chloride (500 μl) was added and roller mixed for a further 50 minutes at 26° C. The solid support was washed a further two times with water (500 μl), roller mixing for 2 minutes in between. Then 1% TFA in water (50 μl) was added to each vial and left for 3 hours at 25° C. roller mixing and a MALDI spectra recorded of the supernatant. The relative integrations of the H₆:D₆ peak at m/z 1681 and m/z 1687 were 1:3.02 and 1:0.21 compared to the theoretical value of 1:4 and 1:0.25.

Synthesis of MalBUT-Sieber-Ficoll

To a solution of the Fmoc-Sieber linker (10 mg, 0.06 mmol) in anhydrous DMSO (500 μl) was added TSTU (17 mg, 0.12 mmol) and diisopropylethylamine (8 μl, 0.12 mmol) and the reaction mixed for 30 minutes. To this solution was added Ficoll PM70 (Amersham Biosciences) (25 mg) and diisopropylethylamine (8 μl, 0.12 mmol) and the mixture agitated. After 2 hours the solution was treated with piperidine (100 μl) to cause removal of the Fmoc protecting group. The solution was diluted to 2.5 ml with deionised water and purified on a Pharmacia PD-10 desalting column. The desired fraction was collected and the water removed in vacuo to afford a white solid of Sieber-Ficoll.

To a solution of maleimidobutyric acid (24 mg, 0.06 mmol) in anhydrous DMSO (500 μl) was added TSTU (17 mg, 0.12 mmol) and diisopyopylethylamine (8 μl, 0.12 mmol). After stirring for 30 minutes the Sieber-Ficoll (see above) and diisopropylethylamine (81 μl, 0.12 mmol) were added and the solution stirred for 2 hours. The solution was diluted to 2.5 ml with deionised water and purified on a Pharmacia PD-10 desalting column.

Cleavage Reaction

To MalBut-Sieber-Ficoll (475 μl) was added TFA (25 μl) and left at 26° C. for 16 hours. 135 μl was injected onto an HPLC, giving a peak at 8.3 mins believed to be maleimidobutyramide. To confirm the presence of the amide, authentic material was produced by addition of MalBut-Sieber-CPG (3.81 mg) to 1% TFA in water (381 μl) and the reaction left for 30 minutes at 26° C. An equimass amount of the cleaved authentic material was mixed with the cleaved MalBut-Sieber-Ficoll solution and the two co-injected onto the HPLC. The putative maleimidobutyramide from acidolytic cleavage of MalBut-Sieber-Ficoll appeared at identical retention time to authentic material.

The Chemical Capture and cleavage from MalBut-Sieber-Ficoll was demonstrated as follows: to MalBut-Sieber-Ficoll (475 μl) was added laminin fragment 925-933 (1 mg/ml in 0.1M phosphate pH 8.0, 50 μl) and left at 26° C. for 20 minutes. The Ficoll was then desalted on a Pharmacia PD-10 column into water, taking 1 minute fractions. 10 μl TFA was added to each fraction and left for 16 hours at 26° C. A MALDI spectra was recorded on each fraction. A peak at 1149.27, corresponding to laminin fragment 925-933 plus maleimidobutyramide, was observed.

Protein Extraction from Escherichia coli

Protein extraction from the control and stressed population is achieved following cell lysis. 50 ml of Escherichia coli cells from an overnight culture of each sample is harvested by centrifugation and the supernatant discarded. The cell pellet produced is dissolved in lysis buffer (10 mM tris pH 8.0, 5 mM magnesium acetate, 8 M urea, 4% CHAPS) and incubated at 4° C. for 1 hour. The lysate is sonicated until the solution becomes clear. The final concentration of protein can be determined using a BCA protein assay (BiORad).

Protein Reduction and Trypsin Digestion

The lysate is reduced by addition of 30 μl of 10 mM tris(2-carboxyethyl)phosphine hydrochloride to 300 μl of each cell lysate and incubated for 30 minutes at 37° C. Both cell lysates are then diluted 1:1 with 100 mM bicarbonate buffer pH 8.0, 0.5 ml total volume to give a protein solution of around 2.5 mg/ml.

Trypsin (100 μg/ml in 1 mM hydrochloric acid, 0.1% (w/v), 12.5 μl) is added to each of the cell lysates above and left at 25° C. for 20 hours. The reaction is quenched by the addition of 2 μl of 0.1 mg/ml Nα-tosyl-L-lysyl chloromethyl ketone (TLCK) as a trypsin inhibitor.

Labelling of Tryptic Digest

Each of the reduced and digested protein lysates (500 μl as above) is added to the solid support synthesised as described above (0.1 g) containing either the deuterium or the proton label at pH 8.0 and the reaction is incubated at 25° C. for 2 hours. The two labelling reactions/resins are then pooled in a single tube for further processing. The excess maleimide functionalities on the resin are quenched by the addition of 0.16 g of 2-mercaptoethanol and incubated at 25° C. for 15 minutes. The resin is washed thoroughly with 0.1 M phosphate pH 7.0 (10 ml) to remove unbound peptides and mercaptoethanol.

Cleavage of Labelled Peptides from Resin and Separation

The labelled peptides are cleaved from the resin by the addition of 5 ml of 1% trifluoroacetic acid in to the pooled resin-bound peptides and incubated at 25° C. for 60 minutes. The supernatant is collected and the resultant peptide mixture injected onto a peptide C₁₈ column pre-equilibrated in 0.1% trifluoroacetic acid/water. The peptides are separated using an increasing gradient of 90% acetonitrile/10% water with 0.1% trifluoroacetic acid over 60 minutes separation rum 1 min (1 ml) fractions are collected.

MS Analysis of Peptide Fractions

The peptide fractions are dissolved in 5 μl of 0.1% trifluoroacetic acid in water followed by the addition of 5 μl of 10 mg/ml α-cyanohydrocinnamic acid in 50:50 acetonitrile:ethanol. The peptide-matrix sample (0.5 μl) is spotted onto a MALDI target plate and the molecular weight of the peptide measured in positive ion reflection mode. The relative integration of all peaks shifted by 4 Da is determined for relative quantification of the component peptides.

1. A capture reagent comprising Formula (I):
PRG-L-X-carrier (I)
wherein:
PRG is a peptide or protein reactive group;
L is a linker group including a stable isotopic detectable label moiety; and
X is a cleavable group.

2. The capture reagent of claim 1, wherein the peptide or protein reactive group is selected from the group consisting of maleimide, N-hydroxysuccinimidyl ester, pyridyldisulfide, vinylsulfone, iodoacetamide, epoxide, nitrile, aryl thiol, sulfonated alkyl, isothiocyanate, isocyanate, imidoester, sulphonyl halide, aldehyde, ketone, amine, alcohol, hydrazine, fluorophosphonate, α-halo methyl ketone, acyloxymethyl ketone and 4-(phenylamino)quinazoline.

3. The capture reagent of claim 2, wherein the peptide or protein reactive group is maleimide.

4. The capture reagent of claim 2, wherein the peptide or protein reactive group is N-hydroxysuccinimidyl ester.

5. The capture reagent 4 of claim 1, wherein the linker L group is selected from the group consisting of ether, polyether, amide, polyamide, polythioether, disulfide, silyl ether, alkyl, alkenyl, aryl, diaryl and alkyl-aryl groups.

6. The capture reagent of claim 5, wherein said linker group L is a C₆ or a C₄ alkyl group.

7. The capture reagent of claim 1, wherein cleavage of the cleavable group results in the detectable label moiety remaining bound to the PRG.

8. The capture reagent of claim 1, wherein said isotopic detectable label moiety is selected from the group consisting of ²H, ¹³C, ¹⁵N, ¹⁷O, ¹⁸O, ³⁴S, ³³S and ³⁶S.

9. The capture reagent of claim 1, wherein said cleavable group is cleavable by treatment with an agent selected from the group consisting of acids, bases and enzymes.

10. The capture reagent of claim 1, wherein the cleavable group is cleavable by means selected from the group consisting of photochemical, thermal, electrochemical, reductive, nucleophilic and electrophilic cleavage.

11. The capture reagent of claim 9, wherein the cleavable group is selected from the group consisting of 3-(aminomethyl)-indol-1-yl-acetic acid, 9(amino-xanthen-3-yl)oxy acetic acid and 4-[amino-(2,4-dimethoxyphenyl)-methyl]-phenol.

12. The capture reagent of claim 1, wherein the carrier is a solid support.

13. The capture reagent of claim 1, wherein the carrier is a water soluble carrier.

14. The capture reagent of claim 13, wherein said water soluble carrier includes Ficoll PM70.

15. A capture reagent of Formula (II):

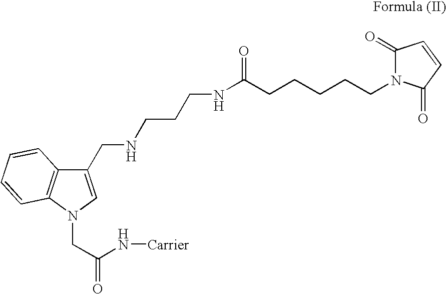

16. A capture reagent of Formula (III)

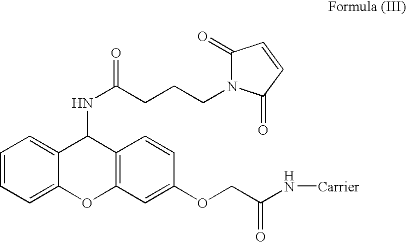

17. A capture reagent of Formula (IV)

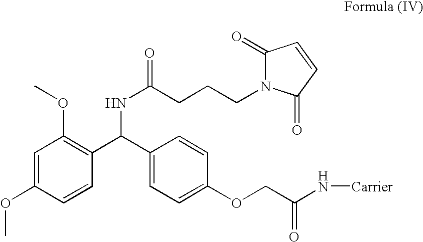

18. (cancelled)

19. A method for selective labelling and purifying of proteins and peptides comprising the steps of
a) providing the capture reagent of claim 1 to a sample containing proteins or peptides under conditions to promote attachment of the proteins or peptides to the PRG;
b) releasing captured components from the carrier; and
c) detecting and identifying the released components.

20. A method for selective labelling and purifying of proteins and peptides in one or more samples containing mixtures of proteins or peptides comprising:
a) providing the capture reagent of claim 1 to one or more samples containing proteins or peptides under conditions to promote attachment of the proteins or peptides to the PRG to result in captured samples, wherein the detectable label of the capture reagent provided to one sample is distinguishable from that provided to another;
b) releasing captured components from the carrier; and
c) detecting and identifying the released components.

21. The method of claim 20, further comprising a pre-treatment step wherein functional groups on the protein or peptide are modified to render them reactive with the protein reactive group prior to conducting step a).

22. The method of claim 21, wherein carboxylic acid groups on the peptide or protein are treated with carbodiimide.

23. The method of claim 21, wherein phosphate groups on the peptide or protein are eliminated to leave a reactive alkene group.

24. The method of claim 21, wherein vicinal diol groups are oxidized or aldehyde or ketone groups in carbohydrate-containing peptides and proteins are dehydrated and reduced.

25. The method of claim 20, further comprising:
d) measuring the relative abundance of the released components derived from each sample.

26. The method of claim 20, wherein the captured samples are mixed prior to releasing the captured components from the carrier.

27. The method of claim 20, further comprising a washing step prior to step b).

28. (cancelled)

29. A kit for use in the analysis of proteomes, said kit comprising the capture reagent of claim 1.

30. An automated system for analysis of proteomes comprising use of the capture reagent of claim 1.
